SEQUENCE LISTING (13 sequences)

SEQ ID NO: 1 - protein, 155 aa, Human
KRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 2 - protein, 29 aa, Artificial
  c29- Fragment of p75ICD
KRWNSCKQNKQGANSRPVNQTPPPEGEKL

SEQ ID NO: 3 - protein, 14 aa, Artificial
  c1-14 - Fragment of p75ICD
KRWNSCKQNKQGAN

SEQ ID NO: 4 - protein, 6 aa, Artificial
  c1-6 - Fragment of p75ICD
KRWNSC

SEQ ID NO: 5 - protein, 15 aa, Artificial
  c15-29 - Fragment of p75ICD
SRPVNQTPPPEGEKL

SEQ ID NO: 6 - protein, 7 aa, Artificial
  c15-21 - Fragment of p75ICD
SRPVNQT

SEQ ID NO: 7 - protein, 40 aa, Artificial
  TAT-c29 - Fusion of TAT with c29
YARAAARNARAKRWNSCKQNKQGANSRPVNQTPPPEGEKL

SEQ ID NO: 8 - protein, 25 aa, Artificial
  TAT-c1-14 - Fusion of TAT and c1-4
YARAAARNARAKRWNSCKQNKQGAN

SEQ ID NO: 9 - protein, 17 aa, Artificial
  TAT-c1-6 - Fusion of TAT and c1-6
YARAAARNARAKRWNSC

SEQ ID NO: 10 - protein, 26 aa, Artificial
  TAT-c15-21 - Fusion of TAT and c15-29
YARAAARNARASRPVNQTPPPEGEKL

SEQ ID NO: 11 - protein, 18 aa, Artificial
  TAT-c 15-21 - Fusion of TAT and c15-21
YARAAARNARASRPVNQT

SEQ ID NO: 12 - protein, 166 aa, Artificial
  TAT-p75ICD - Fusion of TAT and p75ICD
YARAAARNARAKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 13 - protein, 11 aa, Human immunodeficiency virus
YARAAARNARA

(19) United States
(12) Patent Application Publication
(10) Pub. No.: US 2015/0344535 A1
(43) Pub. Date: Dec. 3, 2015

(54) NEUROTROPHIN-TYROSINE KINASE RECEPTOR SIGNALING

(71) Applicant: THE UNIVERSITY OF QUEENSLAND, Queensland (AU)

(72) Inventors: Elizabeth Jane COULSON, St Lucia (AU); Dusan MATUSICA, St. Marys (AU); Timothy William BREDY, Irvine, CA (US); Sune SKELDAL, Aarhus C (DK)

(73) Assignee: THE UNIVERSITY OF QUEENSLAND, Queensland (AU)

(21) Appl. No.: 14/432,190

(22) PCT Filed: Sep. 27, 2013
(86) PCT No.: PCT/IB2013/058910

§ 371 Date: Mar. 27, 2015

(30) Foreign Application Priority Data
Sep. 28, 2012 (AU) 2012904244

(51) Int. Cl. C07K 14/47; G01N 33/566

(52) U.S. Cl. CPC C07K 14/47; G01N 33/566; A61K 38/00

(57) ABSTRACT

The invention relates to a method of regulating neurotrophin-tyrosine kinase receptor signaling in a cell comprising modulating the interaction between the juxtamembrane region of the intracellular domain of the neurotrophin receptor p75 and Trk. The invention also relates to compositions comprising compounds which modulate the interaction between the juxtamembrane region of the intracellular domain of the neurotrophin receptor p75 and Trk, and use of the compounds and compositions for the treatment of neurotrophin-Trk signaling related diseases and disorders.

### FIELD

The present disclosure relates to methods and compositions for regulating neurotrophin-tyrosine kinase receptor signaling in a cell comprising modulating the interaction between the juxtamembrane region of the intracellular domain of the neurotrophin receptor p75 and Trk.

### BACKGROUND

Neurotrophins belong to a small family of secreted growth factors containing nerve growth factor (NGF), brain derived neurotrophic factor (BDNF), neurotrophin-3 (NT-3) and neurotrophin-4 (NT-4) that are important for the generation, migration, maturation, and survival of the neurons that comprise the nervous system. Neurotrophins elicit their effects by binding two structurally unrelated receptors, the common p75 neurotrophin receptor (p75^{NTR}), and one of three tropomysin receptor kinase family members termed TrkA, TrkB and TrkC, each with distinct specificity for particular neurotrophins.

On binding their ligands, p75^{NTR} and Trk receptors can either signal independently of each other or act synergistically. The signalling pathways activated by Trk receptors, which are predominately neurotrophic, have been widely studied and are well defined. However, identification of the components of the signalling pathways downstream of p75^{NTR}, and elucidation of which of these are activated to mediate its wide range of functional activities, has proven difficult, with many of the pathways reported to date remaining controversial or poorly defined.

p75^{NTR} is currently best known for its ability to mediate neurite pruning and neuronal death during development and in neurodegenerative conditions. However, following the identification of Trk receptors, the first characterized function for p75^{NTR} was to facilitate trophic signalling mediated by Trk, assisting neurons to respond to low levels of neurotrophins in vivo and in vitro. p75^{NTR} can cooperate with Trk to either increase the specificity of Trk receptors for a particular ligand, or increase the binding affinity of the neurotrophins for their Trk receptors. For example, although TrkA and p75^{NTR} alone have low-affinity binding rates for NGF, albeit with different on-off dynamics, co-expression of both receptors enables the formation of binding sites with an apparent 30-100 fold higher affinity.

Over the last two decades, a number of models have been proposed to explain the formation of high-affinity binding sites, the principal ones being: (i) the formation of a classic 1:1 heterodimer complex with a 25 fold higher on rate than that of the individual receptors and (ii) the ligand passing model, in which p75^{NTR} (with fast on-off rates) first binds to NGF before releasing the ligand for TrkA to bind (slow on-off rates). However, neither model entirely accounts for the existing experimental data. For example, the formation of a heterodimer is not supported by recent structural studies or by the requirement for TrkA homodimerisation in order to initiate downstream phosphorylation cascades. Furthermore, high-affinity binding can be reconstituted in cells expressing TrkA and a form of p75^{NTR} lacking its ligand-binding domain, provided that p75^{NTR} retains its transmembrane and intracellular juxtamembrane domains. Likewise, the p75^{NTR} homologue NRH2, which also lacks an extracellular NGF binding domain can similarly facilitate TrkA signalling in response to NGF. Therefore, although ligand transfer may occur, it cannot be the sole basis of highaffinity binding, and thus the mechanism by which p75^{NTR} and TrkA generate highaffinity binding sites remains unresolved.

p75^{NTR} receptors lacking the extracellular ligand-binding domain are generated endogenously via proteolytic cleavage. An α-secretase (ADAM17) removes almost the entire extracellular domain, leaving a membrane-bound carboxyterminal fragment (p75^{CTF}), which is subsequently cleaved by γ-secretase, releasing the intracellular domain (p75^{ICD}) into the cytoplasm. This p75^{ICD} fragment has been reported to have important roles in cell survival, neurite outgrowth, differentiation and cell death.

It is an aim of an embodiment of the present disclosure to provide methods and compositions for regulating neurotrophin-tropomyosin kinase receptor signaling in a cell.

### SUMMARY

The inventors of the present disclosure previously reported that in TrkA-expressing neurons, membrane-localised p75^{CTF} constitutively induces cell death mediated by the membrane proximal intracellular juxtamembrane 29 amino acid fragment termed the “Chopper” domain, whereas p75^{ICD}, or a peptide comprising the juxtamembrane fragment lacking a transmembrane linker (c29), instead inhibits neuronal death.

The inventors have now found that this same c29 peptide interacts with the neurotrophic tyrosine kinase receptors, TrkA and TrkB, and acts as a modulator of Trk, facilitating TrkA- and TrkB-mediated signalling, neurite outgrowth and neuronal survival.

Accordingly, in a first aspect, the present disclosure provides a method of regulating neurotrophin-tyrosine kinase receptor (neurotrophin-Trk) signaling in a cell comprising contacting the cell with an agent capable of modulating the interaction between the juxtamembrane region of the intracellular domain of the neurotrophin receptor p75 (p75^{ICD} juxtamembrane region) and Trk.

By modulating the interaction between the p75^{ICD} juxtamembrane region and Trk, it is possible to control the signaling of neurotrophins through Trk receptors. For example, by facilitating the binding of the p75^{ICD} juxtamembrane region to Trk, neurotrophin signaling is increased, while inhibiting the binding of the p75^{ICD} juxtamembrane region to Trk decreases neurotrophin signaling.

The neurotrophin-Trk signaling may comprise any neurotrophin known to signal through the Trk receptors. Signalling through the Trk receptors may include signaling in conjunction with the p75 neurotrophin receptor (p75^{NTR}). In one embodiment, the neurotrophin is selected from nerve growth factor (NGF), brain derived neurotrophic factor (BDNF), neurotrophin-3 (NT-3) and neurotrophin-4 (NT-4). In another embodiment, the tyrosine receptor kinase is selected from TrkA, TrkB and TrkC. In one embodiment, the neurotrophin-Trk signaling is NGF-TrkA signaling. In another embodiment, the neurotrophin-Trk signaling is BDNF-TrkB or NT-4-TrkB signaling. In another embodiment, the neurotrophin-Trk signaling is NT-3-TrkC signaling.

It will be appreciated that the methods described herein may involve modulation of the interaction between p75^{ICD} juxtamembrane region and Trk directly or indirectly. For example, direct modulation may involve facilitating the binding of the p75^{ICD} juxtamembrane region to Trk by providing to a cell a peptide corresponding to the p75^{ICD} juxtamembrane region or, alternatively, providing to a cell a molecule, such as an antibody, that inhibits the binding of the p75^{ICD} juxtamembrane region to Trk. Therefore, in one embodiment, neurotrophin-Trk signaling is up-regulated by contacting a cell with a peptide, a polypeptide or a protein that corresponds to all or part of the p75^{ICD} juxtamembrane region SEQ ID NO: 1, or a functional derivative or homologue thereof. In another embodiment, neurotrophin-Trk signaling is up-regulated by contacting a cell with a peptide comprising, consisting essentially of, or consisting of an amino acid sequence selected from the group consisting of SEQ ID NO: 2, SEQ ID NO: 3, SEQ ID NO: 4, SEQ ID NO: 5, SEQ ID NO:7, SEQ ID NO: 8, SEQ ID NO: 9; SEQ ID NO: 10 and SEQ ID NO: 12. In yet another embodiment, neurotrophin-Trk signaling is up-regulated by contacting a cell with the peptide of SEQ ID NO: 2 or SEQ ID NO: 7.

In one embodiment, neurotrophin-Trk signaling is down-regulated by contacting a cell with (i) an antibody that binds specifically to the p75^{ICD} juxtamembrane region SEQ ID NO: 1 or a functional derivative or homologue thereof; (ii) an antibody that binds specifically to the region of Trk that binds the p75^{ICD} juxtamembrane region or a functional derivative or homologue thereof; (iii) a peptide that binds Trk to disrupt binding of p75^{ICD} with Trk; or (iv) a combination thereof.

Alternatively, indirect modulation of the interaction between the p75^{ICD} juxtamembrane region and Trk may involve increasing or decreasing the endogenous levels of p75^{ICD} juxtamembrane region by modulating an upstream pathway. In one embodiment, the endogenous levels of p75^{ICD} juxtamembrane region in a cell are increased by (i) increasing α-secretase action; (ii) increasing γ-secretase action; or (iii) a combination thereof. In another embodiment, the endogenous levels of p75^{ICD} juxtamembrane region are decreased by (i) decreasing p75^{NTR} α-secretase action; (ii) decreasing p75^{NTR} γ-secretase action; or (iii) a combination thereof. Any p75^{NTR} α-secretase or γ-secretase modulators known in the art may be used and are well known to those skilled in the art. Examples of suitable p75^{NTR} α-secretase and γ-secretase inhibitors are TAPI-2 and compound E, respectively.

The present disclosure also relates to compositions that can be used to modulate neurotrophin-Trk signaling. In a second aspect, the present disclosure provides a composition comprising a peptide comprising, consisting essentially of, or consisting of an amino acid sequence selected from the group consisting of SEQ ID NO: 2, SEQ ID NO: 3, SEQ ID NO: 4, SEQ ID NO: 5, SEQ ID NO: 6, SEQ ID NO:7, SEQ ID NO: 8, SEQ ID NO: 9; SEQ ID NO: 10, SEQ ID NO: 11 and SEQ ID NO: 12.

In a third aspect, the present disclosure provides a composition for use in modulating neurotrophin-Trk signaling comprising a compound that modulates the interaction between p75^{ICD} juxtamembrane region and Trk. In one embodiment, the compound comprises:

a. a peptide, a polypeptide or a protein that corresponds to all or part of the p75^{ICD} juxtamembrane region SEQ ID NO: 1, or a functional derivative or homologue thereof;

b. an antibody that binds specifically to the p75^{ICD} juxtamembrane region SEQ ID NO: 1, or a functional derivative or homologue thereof;

c. an antibody that binds specifically to the region of TrkB that binds the p75^{ICD} juxtamembrane region;

d. a peptide that binds Trk without enhancing signaling and blocks binding of Trk by p75^{ICD};

d. a p75^{NTR} α-secretase and/or p75^{NTR} γ-secretase activator; or

e. a p75^{NTR} α-secretase and/or p75^{NTR} γ-secretase inhibitor.

In one embodiment, the composition comprises a peptide comprising, consisting essentially of, or consisting of an amino acid sequence selected from the group consisting of SEQ ID NO: 2, SEQ ID NO: 3, SEQ ID NO: 4, SEQ ID NO: 5, SEQ ID NO: 6, SEQ ID NO:7, SEQ ID NO: 8, SEQ ID NO: 9; SEQ ID NO: 10, SEQ ID NO: 11 and SEQ ID NO: 12. In another embodiment, the composition comprises the peptide of SEQ ID NO: 2 or SEQ ID NO: 7. In a yet further embodiment, the composition comprises a p75^{NTR} α-secretase and/or p75^{NTR} γ-secretase inhibitor. In a further embodiment, the p75^{NTR} α-secretase and p75^{NTR} γ-secretase inhibitors are selected from TAPI-2 and compound E.

The present disclosure also relates to assays that can be used for identifying compounds that modulate neurotrophin-Trk signaling. Therefore, in a fourth aspect, the present disclosure provides an assay for identifying a compound that modulates neurotrophin-Trk signaling comprising:

(i) providing a neurotrophin-Trk signaling system including Trk and p75^{ICD} juxtamembrane region under conditions which promote neurotrophin-Trk signaling;

(ii) contacting the system with a compound;

(iii) measuring the level of neurotrophin-Trk signaling;

(iv) contacting the neurotrophin-Trk signaling system with a candidate compound;

(v) measuring the level of neurotrophin-Trk signaling in the presence of the candidate compound; and

(vi) comparing the measured level of neurotrophin-Trk signaling in the presence of the candidate compound with the level of neurotrophin-Trk signaling in the absence of the candidate compound, wherein a statistically significant alteration in the level of neurotrophin-Trk signaling in the presence of the candidate agent is indicative of a compound that modulates neurotrophin-Trk signaling.

In a fifth aspect, the present disclosure provides a method of identifying a compound that up-regulates neurotrophin-Trk signaling comprising:

(i) providing a neurotrophin-Trk signaling system including Trk and p75^{ICD} juxtamembrane region, under conditions which promote neurotrophin-Trk signaling;

(ii) contacting the system with a compound;

(iii) measuring the level of neurotrophin-Trk signaling;

(iv) contacting the neurotrophin-Trk signaling system with a candidate compound;

(v) measuring the level of neurotrophin-Trk signaling in the presence of the candidate compound; and

(vi) comparing the measured level of neurotrophin-Trk signaling in the presence of the candidate agent with the level of neurotrophin-Trk signaling in the absence of the candidate compound, wherein a statistically significant increase in the level of neurotrophin-Trk signaling in the presence of the candidate agent is indicative of a compound that up-regulates neurotrophin-Trk signaling.

In a sixth aspect, the present disclosure provides a method of identifying a compound that down-regulates neurotrophin-Trk signaling comprising:

(i) providing a neurotrophin-Trk signaling system including Trk and p75^{ICD} juxtamembrane region, under conditions which promote neurotrophin-Trk signaling;

(ii) contacting the system with a compound;

(iii) measuring the level of neurotrophin-Trk signaling;

(iv) contacting the neurotrophin-Trk signaling system with a candidate compound;

(v) measuring the level of neurotrophin-Trk signaling in the presence of the candidate compound; and

(vi) comparing the measured level of neurotrophin-Trk signaling in the presence of the candidate agent with the level of neurotrophin-Trk signaling in the absence of the candidate compound, wherein a statistically significant decrease in the level of neurotrophin-Trk signaling in the presence of the candidate agent is indicative of a compound that down-regulates neurotrophin-Trk signaling.

In a seventh aspect, the present disclosure provides an assay for identifying a compound that modulates neurotrophin-Trk signaling comprising, comprising:

(i) measuring the level of p75^{ICD} juxtamembrane region in a cell;

(ii) contacting the cell with a candidate compound;

(iii) measuring the level of p75^{ICD} juxtamembrane region in the contacted cell of (ii); and

(iv) comparing the measured level of p75^{ICD} juxtamembrane region of (i) and (iii), wherein a statistically significant difference in the level is indicative of a compound that modulates neurotrophin-Trk signaling comprising.

In one embodiment, the neurotrophin-Trk signaling system comprises cells that endogenously express a Trk which is activated by a neurotrophin and treating said cells with a neurotrophin and c29. In another embodiment, the neurotrophin-Trk signaling system comprises cells that are transfected with a construct comprising a gene encoding a Trk which is activated by a neurotrophin and treating said cells with a neurotrophin and c29.

In one embodiment, the neurotrophin-Trk signaling system is a BDNF-TrkB signaling system, wherein said system comprises cells that endogenously express TrkB and treating said cells with BDNF and c29. Cells that endogenously express TrkB include but are not limited to NSC34 motor neuron-like cells, cortical neurons, hippocampal neurons, cerebella granule neurons, glioma cells lines, SH-SY5Y cells.

In another embodiment, the neurotrophin-Trk signaling system is a NGF-TrkA signaling system, wherein said system comprises cells that endogenously express TrkA and treating said cells with NGF and c29. Cells that endogenously express TrkA include but are not limited to PC12 cells, sympathetic, sensory, trigeminal, and basal forebrain neurons.

The compounds disclosed herein may be used to treat or prevent conditions that are mediated by neurotrophin-Trk signalling. Therefore, in an eighth aspect the present disclosure provides a method of treating or preventing a neurotrophin-Trk signaling related disease or disorder in a subject in need thereof, said method comprising administering to said subject a therapeutically effective amount of a composition that comprises a compound that modulates the interaction between p75^{ICD} juxtamembrane region and Trk. In alternative embodiments, there is provided use of a compound that modulates the Interaction between p75^{ICD} juxtamembrane region and Trk compound in the manufacture of a medicament for treating treating or preventing a neurotrophin-Trk signaling related disease or disorder in a subject in need thereof, and a compound that modulates the interaction between p75^{ICD} juxtamembrane region and Trk compound for use in treating treating or preventing a neurotrophin-Trk signaling related disease or disorder in a subject in need thereof. In one embodiment, the method comprises administering to said subject a therapeutically effective amount of a composition that comprises a compound that up-regulates the interaction between p75^{ICD} juxtamembrane region and Trk compound. In one embodiment, a compound that up-regulates the interaction between p75^{ICD} juxtamembrane region and Trk compound is a compound that mimics, or substantially mimics, the binding of the p75^{ICD} juxtamembrane region to the Trk compound. In one embodiment, the compound that up-regulates the Interaction between p75^{ICD} juxtamembrane region and Trk compound is a peptide, polypeptide or protein that corresponds to all or part of the p75^{ICD} juxtamembrane region SEQ ID NO: 1, or a functional derivative or homologue thereof. In one embodiment, the composition comprises a peptide comprising, consisting essentially of, or consisting of an amino acid sequence selected from the group consisting of SEQ ID NO: 2, SEQ ID NO: 3, SEQ ID NO: 4, SEQ ID NO: 5, SEQ ID NO:7, SEQ ID NO: 8, SEQ ID NO: 9; SEQ ID NO: 10 and SEQ ID NO: 12. In another embodiment, the composition comprises the peptide of SEQ ID NO: 2 or SEQ ID NO: 7.

In one embodiment, the method comprises administering to said subject a therapeutically effective amount of a composition that comprises a compound that down-regulates the interaction between p75^{ICD} juxtamembrane region and Trk compound. In one embodiment, the compound that down-regulates the interaction between p75^{ICD} juxtamembrane region and Trk compound is selected from (a) an antibody that binds specifically to the p75^{ICD} juxtamembrane region; (b) an antibody that binds specifically to the region of TrkB that binds the p75^{ICD} juxtamembrane region; (c) a p75^{NTR} α-secretase and/or p75^{NTR} γ-secretase inhibitor; or (d) a combination thereof. In a further embodiment, the p75^{NTR} α-secretase and p75^{NTR} γ-secretase inhibitors are selected from TAPI-2 and compound E.

In one embodiment, the neurotrophin-Trk signaling related disease or disorder is a BDNF-TrkB signaling related disease or disorder, an NT4-TrkB signaling related disease or disorder, a NGF-TrkA signaling related disease or disorder, or an NT3-TrkC signaling related disease or disorder.

In one embodiment, the neurotrophin-Trk signaling related disease or disorder is a psychiatric disorder, such as in schizophrenia, depression and other mood disorders, such as bipolar spectrum disorder. Other psychiatric disorders include anxiety, drug addiction, obsessive-compulsive disorder and Autism spectral disorder. Accordingly, in one embodiment, the neurotrophin-Trk signaling related disease or disorder is a psychiatric disorder. The psychiatric disorder may be anxiety, schizophrenia, depression, bipolar spectrum disorder, drug addiction, obsessive-compulsive disorder and Autism spectral disorder.

In a ninth aspect, the present disclosure provides a method of treating a neurotrophin-Trk signaling related disease or disorder in a subject in need thereof, said method comprising administering to said subject a therapeutically effective amount of a composition comprising a compound that up-regulates the interaction between p75^{ICD} juxtamembrane region and Trk. In alternative embodiments, the present disclosure provides use of a compound that up-regulates the interaction between p75^{ICD} juxtamembrane region and Trk in the manufacture of a medicament for treating a neurotrophin-Trk signaling related disease or disorder in a subject in need thereof, or a compound that up-regulates the interaction between p75^{ICD} juxtamembrane region and Trk for use in treating a neurotrophin-Trk signaling related disease or disorder in a subject in need thereof.

The process of memory extinction, which assists in the removal or rewriting of a memory, including a fear memory, is mediated by neurotrophin-Trk signaling. Accordingly, in a tenth aspect, the present disclosure provides a method of promoting memory acquisition or extinction in a subject in need thereof, said method comprising administering to said subject a therapeutically effective amount of a composition comprising a compound that up-regulates the interaction between p75^{ICD} juxtamembrane region and TrkB. In an eleventh aspect, the present disclosure provides a method of treating a fear-related disorder in a subject in need thereof, said method comprising administering to said subject a therapeutically effective amount of a composition comprising a compound that up-regulates the interaction between p75^{ICD} juxtamembrane region and TrkB. In alternative embodiments, the present disclosure provides:

use of a compound that up-regulates the interaction between p75^{ICD} juxtamembrane region and Trk in the manufacture of a medicament for: promoting memory acquisition or extinction in a subject in need thereof; or treating a fear-related disorder in a subject in need thereof;

or a compound that up-regulates the interaction between p75^{ICD} juxtamembrane region and Trk for use in: promoting memory acquisition or extinction in a subject in need thereof; or treating a fear-related disorder in a subject in need thereof.

In one embodiment, the composition comprises a peptide comprising, consisting essentially of, or consisting of an amino acid sequence selected from the group consisting of SEQ ID NO: 2, SEQ ID NO: 3, SEQ ID NO: 4, SEQ ID NO: 5, SEQ ID NO:7, SEQ ID NO: 8, SEQ ID NO: 9; SEQ ID NO: 10 and SEQ ID NO: 12. In one embodiment, the composition comprises a polypeptide of SEQ ID NO: 2 or SEQ ID NO: 7.

In a twelfth aspect, the present disclosure provides a method of treating a fear-related disorder in a subject in need thereof, said method comprising administering to said subject a therapeutically effective amount of a composition comprising a polypeptide of SEQ ID NO: 2 or 7. In alternative embodiments, there is provided use of a composition comprising a polypeptide of SEQ ID NO: 2 or 7 in the manufacture of a medicament for treating a fear related disorder in a subject in need thereof, or a composition comprising a polypeptide of SEQ ID NO: 2 or 7 for use in treating a fear-related disorder in a subject in need thereof. In one embodiment, the fear-related disorder is selected from post-traumatic stress disorder and panic attacks.

Enhancement of neurotrophin-Trk signaling may also be advantageous for conditions which may benefit from cognitive enhancement. Conditions which may benefit from cognitive enhancement include cerebral palsy, trauma induced paralysis, vascular ischaemia associated with stroke, neural tumours, motor neurone disease, Parkinson's disease, Huntington's disease, Alzheimer's disease, multiple sclerosis. Accordingly, in a thirteenth aspect, the present disclosure provides a method of treating one or more diseases or disorders selected from the group consisting of cerebral palsy, trauma induced paralysis, vascular ischaemia associated with stroke, neural tumours, motoneurone disease, Parkinson's disease, Huntington's disease, Alzheimer's disease, multiple sclerosis, comprising administering to said subject a therapeutically effective amount of a composition comprising a compound that up-regulates the interaction between p75^{ICD} juxtamembrane region and Trk. In alternative embodiments, the present disclosure provides use of a compound that up-regulates the interaction between p75^{ICD} juxtamembrane region and Trk in the manufacture of a medicament for treating the abovementioned diseases or disorders in a subject in need thereof, or a compound that up-regulates the interaction between p75^{ICD} juxtamembrane region and Trk for use in treating the abovementioned diseases or disorders in a subject in need thereof.

Neurotrophin signaling promotes neurite outgrowth and as such compounds that up-regulate neurotrophin-Trk signaling would be useful for treating neurodegenerative injuries and disorders. Accordingly, in one embodiment, the neurotrophin-Trk signaling related disease or disorder is selected from peripheral neuropathies associated with diabetes, heavy metal or alcohol toxicity, renal failure and/or infectious diseases such as herpes, rubella, measles, chicken pox, HIV and/or HTLV-1, chemotherapy, trauma induced paralysis, vascular ischaemia associated with stroke, neural tumours, motor neurone disease, Parkinson's disease, Huntington's disease, Alzheimer's disease, multiple sclerosis.

In a fourteenth aspect, the present disclosure provides a method of promoting neurite outgrowth in a subject in need thereof, said method comprising administering to said subject a therapeutically effective amount of a composition capable of up-regulating neurotrophin-Trk signaling. In alternative embodiments, the present disclosure provides use of a compound that up-regulates neurotrophin-Trk signaling in the manufacture of a medicament for promoting neurite outgrowth in a subject in need thereof, or a compound that up-regulates neurotrophin signaling for use in promoting neurite outgrowth in a subject in need thereof.

A fifteenth aspect provides a method of treating or preventing pain in a subject in need thereof, comprising administering to the subject an effective amount of a composition capable of down-regulating neurotrophin-Trk signaling. In alternative embodiments, the present disclosure provides use of a compound that down-regulates neurotrophin-Trk signaling in the manufacture of a medicament for treating or preventing pain in a subject in need thereof, or a compound that down-regulates neurotrophin-Trk signaling for use in treating or preventing pain in a subject in need thereof.

In a sixteenth aspect, the present disclosure provides a cognitive enhancer comprising a peptide, polypeptide or protein that corresponds to all or part of the p75^{ICD} juxtamembrane region SEQ ID NO: 1. In one embodiment, the cognitive enhancer is a peptide comprising, consisting essentially of, or consisting of an amino acid sequence selected from the group consisting of SEQ ID NO: 2, SEQ ID NO: 3, SEQ ID NO: 4, SEQ ID NO: 5, SEQ ID NO: 6, SEQ ID NO:7, SEQ ID NO: 8, SEQ ID NO: 9; SEQ ID NO: 10, SEQ ID NO: 11 and SEQ ID NO: 12. In another embodiment, the cognitive enhancer is the peptide of SEQ ID NO: 2 or SEQ ID NO: 7.

In one embodiment, the cognitive enhancer up-regulates neurotrophin-Trk signaling. Accordingly, a cognitive enhancer that up-regulates neurotrophin-Trk signaling would be useful in the treatment of disorders that involve a reduction in neurotrophin-Trk signaling. In one embodiment, cognitive enhancer used for the treatment of a disorder selected from post-traumatic stress disorder, panic attacks, schizophrenia, depression, bipolar spectrum disorder, anxiety, drug addiction, obsessive-compulsive disorder and Autism spectral disorder, cerebral palsy, trauma induced paralysis, vascular ischaemia associated with stroke, neural tumours, motor neurone disease, Parkinson's disease, Huntington's disease, Alzheimer's disease, multiple sclerosis.

In a seventeenth aspect, the present disclosure provides a cognitive enhancer that up-regulates neurotrophin-Trk signaling, wherein said cognitive enhancer comprises SEQ ID NO: 2 or SEQ ID NO: 7.

It will be appreciated that the present disclosure also relates to methods of diagnosing diseases or disorders mediated by neurotrophin-Trk signaling. Accordingly, in a sixteenth aspect, the present disclosure provides a method of diagnosing a neurotrophin-Trk signaling related disease or disorder in a subject suspected of having a neurotrophin-Trk signaling related disease or disorder, said method comprising detecting the level of expression of a gene encoding c29 polypeptide (a) in a test sample of tissue cells obtained from a subject, and (b) in a control sample of known normal tissue cells of the same cell type, wherein a higher or lower level of expression of said gene in the test sample as compared to the control sample is indicative of the presence of neurotrophin-Trk signaling related disease or disorder in a subject from which the test tissue cells were obtained.

### BRIEF DESCRIPTION OF THE FIGURES

FIG. 1: Proposed model of neurotrophin-Trk signaling.

FIG. 2: Schematic of mutant p75^{NTR} constructs. p75^{FL}—full-length p75^{NTR}; p75^{CTF}-p75^{NTR} c-terminal fragment (extracellular domain deletion); p75^{TM}^{_{—}}^{JUX}-p75^{NTR} minimal transmembrane fragment and juxtamembrane domain; p75^{ICD}-p75^{NTR} intracellular domain (extracellular and transmembrane domain deletion); p75^{ΔJUX}-p75^{NTR} with a 29 amino acid intracellular juxtamembrane deletion; p75^{NGLY}—full-length p75^{NTR} with a N-linked glycan attachment site which prevents α-cleavage of the receptor (CRD's—cysteine rich domains; TMD—transmembrane domain; JUX—juxtamembrane domain).

FIG. 3: p75^{CTF} promotes cell death whereas p75^{ICD} promotes neuritre outgrowth. (A) Photographs of PC12 cells transfected with control YFP, full-length wild-type and mutant or truncated p75^{NTR}-YFP, and TrkA expression plasmids 5 days after transfection and treatment with 100 ng/ml NGF. Scale bars are 50 μm (B) Quantification of neurite length per transfected PC12 cell per condition for each construct 5 days after NGF treatment (n>50 cells per condition, from 3 experiments; median ±SEM; ***p<0.001; ANOVA, compared to YFP control). (C) Representative Western blots of lysates of transfected PC12 cells 24 hours after treatment with NGF using pErk1/2, total Erk1/2 (tErk) or βIII-tubulin as a loading control. (D) Quantification of band densitometry of pErk1/2 from 3 experiments. (mean±SEM; *p<0.05; **p<0.01; ***p<0.001; ANOVA, compared to p75^{FL}; UT=untreated) (E) Quantification of neurite length per PC12 cell 5 days after treatment with the α- and γ-secretase inhibitors TAPI-2 or compound E, respectively, and either 100 ng/ml NGF (left) or 100 ng/ml EGF (right) treatment (n>50 cells per condition from 3 experiments; median ±SEM; ***p<0.001; ANOVA, compared to untreated control; No GF=no growth factor; UT=untreated).

FIG. 4: TrkA interacts with the intracellular juxtamembrane domain of p75^{NTR}. (A) Co-immunoprecipitation of TrkA by co-expressed p75^{FL}-YFP using anti-green fluorescent protein antibody to either the YFP tag (A) or to the native p75^{NTR} intracellular domain (B, top). Neither the ability of TrkA to be activated, nor the presence of NGF altered the amount of TrkA that co-precipitated with p75^{NTR}. IP: antibody used for immunoprecipitation; WB: antibody used for Western blotting (B) Co-immunoprecipitation of TrkA and TrkAK538A following precipitation of YFP in lysates from cells co-expressing p75^{CTF}-YFP, p75^{TM-JUX}-YFP or p75^{ICD}-YFP proteins with or without NGF. (C) Co-immunoprecipitation of TrkA and TrkAK538A following pull-down of YFP in lysates co-expressing p75^{FL}-YFP or p75^{TM-JUX}-YFP with or without NGF. Neither TrkA nor TrkAK538A is co-immunoprecipitated when p75^{TM-JUX}-YFP is used as the bait. (D) Western blot analysis following pull down using streptavidin-linked beads alone, biotinylated c29 peptide, or a control biotinylated peptide encompassing the extracellular juxtamembrane domain of p75^{NTR} (LC1). Lysates containing TrkA and EGF receptors were derived from transfected HEK293 cells, whereas NSC-34 cells endogenously express TrkB. (E) Sequence of the Intracellular juxtamembrane domain of p75^{NTR}. The transmembrane domain (TMD) is shaded black.

FIG. 5: The c29 peptide enhances NGF-mediated differentiation and survival following NGF withdrawal in PC12 cells. (A) Differential interference contrast (DIC) micrographs of PC12 cells following differentiation in the presence of various NGF concentrations and treatment with c29 or a scrambled control (SC) peptide linked to PTD4 five days after “priming” in 10 ng/ml NGF. Size bars are 50 μM. (B) Quantification of neurite length per PC12 cell per condition 5 days after NGF treatment (n>50 cells per condition from 3 experiments; median ±SEM; *p<0.01 ***p<0.001; ANOVA). (C) Quantification of neurite length per PC12 cell per condition 5 days after EGF treatment (n>50 cells per condition from 3 experiments; median ±SEM; ***p<0.001; ANOVA). (D) Quantification of cell viability (acid phosphatase assay) per well 3 days after medium containing 100 ng/ml NGF was replaced with medium containing NGF at the concentration indicated. (n=8 wells over 4 independent experiments. Mean±SEM; *p<0.01 ***p<0.001; ANOVA).

FIG. 6: c29 enhances pErk1/2 and pAkt signalling in PC12 cells. (A) Representative Western blots of PC12 cell lysates 24 hours after treatment with NGF and/or c29 or scrambled (SC) peptide and probed with Erk1/2 and Akt antibodies as indicated. (B) Quantification of band densitometry of pErk1/2 (top) and pAkt (bottom) Western blots. (Mean±SEM, n=3 experiments; *p<0.05; ***p<0.001; ANOVA, compared to 10 ng/ml NGF control treatment).

FIG. 7: c29 peptide acts synergistically with TrkB to enhance the responsiveness of motor neurons to BDNF. (A) Percentage of primary E13 motor neurons surviving for 3 days in culture in various concentrations of BDNF (n=3 experiments; mean±SEM; ***p<0.001; ANOVA). (B) Percentage of motor neurons surviving 24 hours after the reduction in concentration of growth factor as indicated in the culture medium in which they were plated. (n=3 experiments; mean±SEM; *p<0.01; ANOVA). (C) DIC micrographs of motor neurons cultured for 3 days in the presence of 1 ng/ml BDNF or 1 ng/ml BDNF and 50 ng/ml NGF with or without c29 treatment. Scale bar represents 50 μm. (D) Percentage of motor neurons surviving 3 days in the presence of 1 ng/ml BDNF or BDNF and 50 ng/ml NGF, with or without c29 treatment (n=3 experiments; mean±SEM; ***p<0.001; ANOVA). (E) Percentage decrease in four day old rat cervical motor neuron number 3 days following unilateral axotomy of the ulnar nerve, relative to contralateral motor neuron number. PTD4-c29 but not PTD4 peptides resulted in significant protection from motor neuron loss. (n=5 animals per group, mean±SEM; ***p<0.0011 tailed t-test). (F) Micrographs of cresyl violet-stained, non-lesioned (left) cervical spinal cord sections of four day old rats 5 days after unilateral axotomy of the ulnar nerve with (right) or without (middle) application of c29 peptide-soaked gel foam to the axonal stump.

FIG. 8: Graph of motor neuron survival (Y-axis) in 115 day postnatal SOD1G93A mutant mice following systemic treatment at 60 days postnatal with 5 mg/kg of TAT-c29 (SEQ ID NO: 7) (black column) or saline (white column).

FIG. 9: c29 alters the binding of NGF to cells expressing TrkA. (A) Representative flow cytometry plots of population fluorescence of HEK293 cells co-transfected with TrkAK538A and p75^{FL} and treated with 26 nM NGF-FITC for 60 minutes. Control is non-transfected cells treated with 26 nM NGF-FITC. (B) Representative flow cytometry plots of population fluorescence of TrkAK538A expressing HEK293 cells co-transfected with p75^{FL} or p75^{ΔJUX} treated with NGFFITC for 60 minutes. Control is non-transfected cells treated with NGF-FITC. (C) Graph of NGF-binding competition curves HEK293 cells co-transfected with TrkAK538A and p75^{FL} or p75^{ΔJUX} (n=3 experiments, mean fluorescence of the population standardised to the no competition condition (100%)) (D) Representative flow cytometry plots of the population fluorescence of TrkAK538A-transfected HEK293 cells with or without c29 peptide pre-treatment with NGF-FITC for 60 minutes. Control is non-transfected c29-treated cells with NGF treatment. (E) Graph of the Hill-Slope association rates of FITC-NGF to HEK293 cells transfected with p75^{FL} with or without pre-treatment with c29. The lines are calculated from the mean population fluorescence over time of flow cytometry events recorded across 3 experiments. (F) Graph of the Hill-Slope association rates of FITC-NGF to HEK293 cells transfected with TrkAK538A with or without pre-treatment with c29. The lines are calculated from the mean population fluorescence over time of flow cytometry events recorded across 3 experiments.

FIG. 10: The juxtamembrane domain of p75^{NTR} increases the association of NGF to cells expressing TrkA. (A and B) Dose response graphs of the proportion (%) of PC12 cells with fluorescence intensity above background following treatment with 26 ng/ml NGFFITC, a FITC-conjugated antibody to the extracellular domain of p75^{NTR} (A), or an antibody to TrkA (detected using an anti-rabbit Alexa 594 secondary) (B), after pretreatment with increasing concentrations of c26 peptide. (n=3 experiments per data point; mean±SEM *p<0.05, **p<0.01) (C) Representative western blots (WB) of TrkA and p75^{NTR} present in streptavidin precipitates (surface biotin), or lysate flow through of PC12 cells that had been treated for 1 hour with NGF and/or c29 and scrambled (SC) peptide, prior to the surface proteins being labelled with biotin for 90 minutes on ice.

FIG. 11: A: Graph showing memory acquisition (freeze response) at prestimulus (PCS), conditioned stimulus 1 (CS1), conditioned stimulus 2 (CS2) and conditioned stimulus 3 (CS3) in mice treated by an infusion of TAT-c29 (c29), scrambled peptide (SC) or vehicle (naïve) into the prefrontal cortex 1 hour prior to fear conditioning training. B: A graph of the average of the response to preconditioned stimulus (PCS) and post conditioned stimulus (AvgCS) as measured by freezing behavior 24 hours after conditioning in mice treated by an infusion of TAT-c29 (c29), scrambled peptide (SC) or vehicle (naïve) into the prefrontal cortex.

FIG. 12: Local infusion of c29 into the infralimbic prefrontal cortex blocked the return of fear in female mice when tested in the context in which was acquired (n=5/group; t-test VEH RET vs C29 RET, p<0.05).

FIG. 13: Graph showing excitatory postsynaptic potential in the hippocampus over time in mice 60 minutes after systemic administration of a single 10 mg/kg i.p. injection of TAT-c29 (5 or 10 mg/kg as indicated), scrambled peptide (10 mg/kg) or C57.

FIG. 14 is a western blot showing enhanced trophic signaling (phosphorylated Erk1/2) in response to BDNF in TrkB-expressing cortical neurons when c29, but not scrambled peptide (sc), is applied. c29 enhances Erk1/2 phosphorylation in E16 cultured cortical neurons 15 minutes after BDNF treatment.

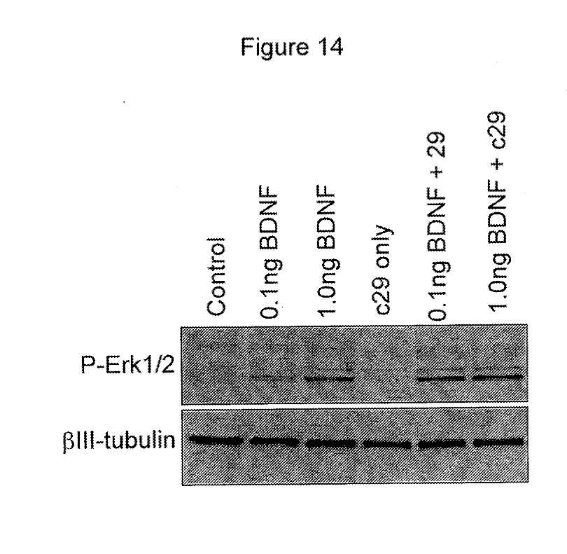

FIG. 15: Functional fragments of c29. (A) Western blot probed with TrkA antibody. Lysate was from 293 cells which do not normally express Trk or p75 receptors, genetically transfected with TrkA expression vector. (B) Western blot probed with TrkA antibody. Lysate was from PC12 cells which normally (endogenously) express TrkA and p75 receptors. (C) Western blot probed with TrkB antibody. Lysate was from 293 cells which do not normally express Trk or p75 receptors, genetically transfected with TrkB expression vector. (D) Western blot of probed with TrkB antibody. Lysate was from NSC34 cells which normally (endogenously) express TrkB and p75 receptors. (E) Schematic diagram of various truncated peptides used based on c29 (top) with an indication of the extent of TrkA binding capacity. (F) Western blot of TrkA pull downs using the various biotinylated peptides.

FIG. 16: Graph of neurite outgrowth in response to NGF in PC12 cells treated with c29 variants.

### DETAILED DESCRIPTION OF THE PREFERRED EMBODIMENTS

Before describing the present disclosure in detail, it is to be understood that this disclosure is not limited to particularly exemplified cell culture techniques, serum, media or methods and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

The practice of the present invention will employ, unless otherwise indicated, conventional techniques of cell biology, cell culture and molecular biology, which are within the skill of the art. Such techniques are described in the literature. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

By “comprising” is meant including, but not limited to, whatever follows the word comprising”. Thus, use of the term “comprising” Indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

It must be noted that as used herein and in the appended claims, the singular forms “a”, “an”, and “the” include plural reference unless the context clearly dictates otherwise. Thus, for example, a reference to “a polypeptide” includes a plurality of such polypeptides, and a reference to “an antibody” is a reference to one or more antibodies, and so forth.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

In the broadest sense, the present invention relates to a method of regulating neurotrophin-Trk signaling. The term “neurotrophin-Trk signaling”, as used herein, means the activation of biochemical pathways triggered by the interaction of a neurotrophin with a tyrosine receptor kinase (Trk). Accordingly, the “regulation of neurotrophin-Trk signalling” refers to increasing (“up-regulation”) and decreasing (“down-regulation”) the activation of such pathways. Methods of determining whether neurotrophin-Trk signaling is up- or down-regulated can be readily determined by a person skilled in the art and are discussed infra.

The neurotrophin-Trk signaling may comprise any neurotrophin known to signal through the Trk receptors. Signalling through the Trk receptors may include signaling in conjunction with the p75 neurotrophin receptor (p75^{NTR}). Examples of neurotrophins that signal through Trk include nerve growth factor (NGF), brain derived neurotrophic factor (BDNF), neurotrophin-3 (NT-3) and neurotrophin-4 (NT-4). Examples of tyrosine receptor kinase that bind neurotrophins include TrkA, TrkB and TrkC. In one embodiment, the neurotrophin-Trk signaling is NGF-TrkA signaling. In another embodiment, the neurotrophin-Trk signaling is BDNF-TrkB signaling.

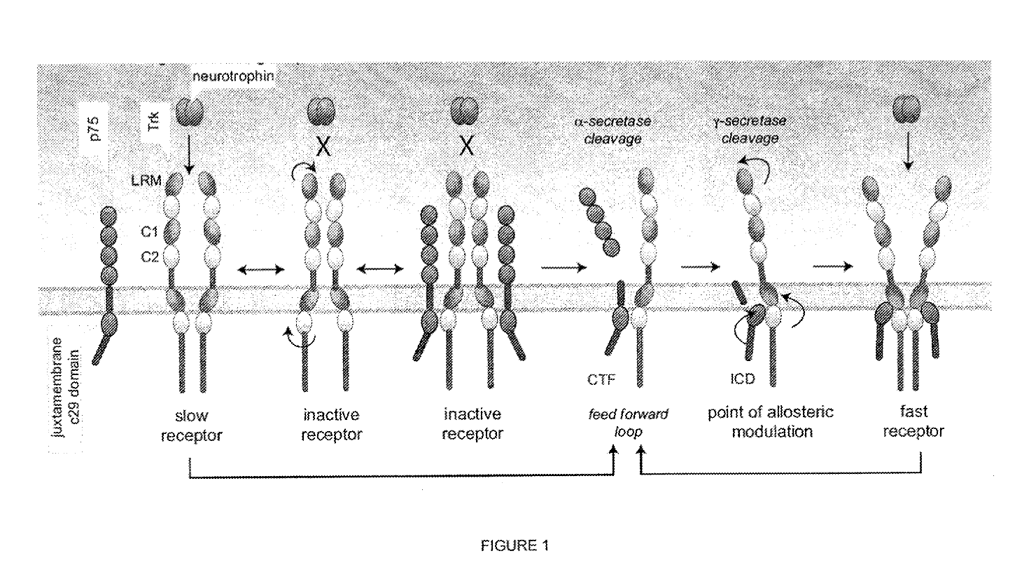

Neurotrophin-Trk signaling can be up- or down-regulated by modulating the interaction between the juxtamembrane region of the intracellular domain of the neurotrophin receptor p75 (“p75^{ICD} juxtamembrane region”) and Trk. Without wishing to be bound by any particular theory or hypothesis, the inventors of the present disclosure believe that the endogenous p75^{NTR} intracellular domain fragment interacts with Trk, and acts as a modulator of Trk, facilitating Trk-mediated signaling. It is believed that the affinity of Trk for neurotrophins is modulated by an inside-out allosteric mechanism mediated by the juxtamembrane domain of p75^{NTR}. A model of how high-affinity binding sites are generated is provided in FIG. 1. A key element of the model is that p75^{NTR} must be processed by secretases to generate an intracellular domain fragment (p75^{ICD}) prior to being able to modulate Trk function. Therefore, by modulating the interaction between the p75^{ICD} juxtamembrane region and Trk, neurotrophin-Trk signaling can be regulated.

The term “modulation”, “modulating”, and grammatical equivalents thereof, means to affect the interaction between the p75^{ICD} juxtamembrane region and Trk such that neurotrophin-Trk signaling is modified. “Modulating” includes: (a) up-regulating neurotrophin-Trk signaling; and (b) down-regulating neurotrophin-Trk signaling. Up-regulating of neurotrophin-Trk signaling may be, for example, by facilitating the binding of the p75^{ICD} juxtamembrane region to Trk. A compound that facilitates the binding of the p75^{ICD} juxtamembrane region to Trk includes a compound that mimics or substantially mimics the binding of the p75^{ICD} juxtamembrane region to Trk. Thus, a compound that modulates the interaction between p75^{ICD} juxtamembrane region and Trk includes a compound that mimics, or substantially mimics, the binding of the p75^{ICD} juxtamembrane region to Trk. A compound that “mimics or substantially mimics” refers to a compound that binds in a manner that produces the same, or substantially the same, biological effect. Down-regulating of the neurotrophin-Trk signaling may be, for example, by inhibiting the binding of the p75^{ICD} juxtamembrane region to Trk.

It will be appreciated that the methods described herein may involve modulation of the interaction between p75^{ICD} juxtamembrane region and Trk directly or indirectly. For example, direct modulation may involve facilitating the binding of the p75^{ICD} juxtamembrane region to Trk by providing to a cell a peptide corresponding to the p75^{ICD} juxtamembrane region or, alternatively, providing to a cell a molecule, such as an antibody or peptide, that inhibits the binding of the p75^{ICD} juxtamembrane region to Trk.

In one embodiment, neurotrophin-Trk signaling is directly up-regulated by contacting a cell with an isolated peptide, polypeptide or protein that corresponds to all or part of the p75^{ICD} juxtamembrane region. Preferably, the peptide, polypeptide or protein comprises an amino acid sequence substantially as set forth in SEQ ID NO: 1 or an amino acid sequence having at least 60% identity thereto or a functional derivative, homologue or analogue of said peptide, polypeptide or protein.

p75^{ICD}
SEQ ID NO: 1
KRWNSCKQNKQGANSRPVNQTPPPEGEKLHSDSGISVDSQ
SLHDQQPHTQTASGQALKGDGGLYSSLPPAKREEVEKLLN
GSAGDTWRHLAGELGYQPEHIDSFTHEACPVRALLASWAT
QDSATLDALLAALRRIQRADLVESLCSESTATSPV

The term “isolated” means that the peptide, polypeptide or protein is provided in a form which is distinct from that which occurs in nature, preferably wherein one or more contaminants have been removed. Accordingly, the isolated peptide, polypeptide or protein may be partially-purified or substantially pure, in which a substantial amount of the contaminants have been removed or substantially homogeneous form.

The term “substantially homogeneous” means that the isolated peptide, polypeptide or protein of the present invention is at least about 95% free of contaminants, more preferably at least about 99% free of contaminants, including 100% purity.

The present invention extends to a range of derivatives and chemical analogues of the peptide, polypeptide or protein.

Furthermore, the amino acids of a homologous polypeptide may be replaced by other amino acids having similar properties, for example hydrophobicity, hydrophilicity, hydrophobic moment, charge or antigenicity, and so on.

The term “derivative” in relation to a peptide, polypeptide or protein shall be taken to refer hereinafter to mutants, parts or fragments derived from the functional p75^{ICD} juxtamembrane region thereof or derivatives thereof. A “functional p75^{ICD} juxtamembrane region” is a peptide, polypeptide or protein capable of interacting with Trk to produce an active receptor. Examples of functional derivatives of p75^{ICD} juxtamembrane region include peptides selected from the following:

| | | |
|---|---|---|
| c29 (1-29) | KRWNSCKQNKQGANSRPVNQTP | (SEQ ID NO: 2) |
| | PPEGEKL | |
| (1-14) | KRWNSCKQNKQGAN | (SEQ ID NO: 3) |
| (1-6) | KRWNSC | (SEQ ID NO: 4) |
| (15-29) | SRPVNQTPPPEGEKL | (SEQ ID NO: 5) |
| (15-21) | SRPVNQT | (SEQ ID NO: 6) |
| TAT-c29 | YARAAARNARAKRWNSCKQNKQ | (SEQ ID NO: 7) |
| | GANSRPVNQTPPPEGEKL | |
| TAT-1-14 | YARAAARNARAKRWNSCKQNKQ | (SEQ ID NO: 8) |
| | GAN | |
| TAT-1-6 | YARAAARNARAKRWNSC | (SEQ ID NO: 9) |
| TAT-15-29 | YARAAARNARASRPVNQTPPPE | (SEQ ID NO: 10) |
| | GEKL | |
| TAT-15-21 | YARAAARNARASRPVNQT | (SEQ ID NO: 11) |
| TAT-p75^{ICD} | YARAAARNARAKRWNSCKQNKQ | (SEQ ID NO: 12) |
| | GANSRPVNQTPPPEGEKLHSDS | |
| | GISVDSQSLHDQQPHTQTASGQ | |
| | ALKGDGGLYSSLPPAKREEVEK | |
| | LLNGSAGDTWRHLAGELGYQPE | |
| | HIDSFTHEACPVRALLASWATQ | |
| | DSATLDALLAALRRIQRADLVE | |
| | SLCSESTATSPV | |

Derivatives also include modified peptides in which ligands are attached to one or more of the amino acid residues contained therein, such as carbohydrates, enzymes, proteins, polypeptides or reporter molecules such as radionucleotides, fluorescent compounds, or biotin. Procedures for derivatizing peptides are well-known in the art.

“Analogues” encompass peptides, polypeptides or proteins which are at least about 60% identical to the p75^{ICD} juxtamembrane region amino acid sequence substantially as set forth in SEQ ID NO: 1, notwithstanding the occurrence of any non-naturally occurring amino acid analogues therein. “Analogues” also encompass polypeptide mimotypes.

A homologue, analogue or derivative of may comprise an amino acid substitution or encompass amino acid alterations in which an amino acid is replaced with a different naturally-occurring or a non-conventional amino acid residue. Such substitutions may be classified as “conservative”, in which case an amino acid residue contained in a phospholipase inhibitory protein is replaced with another naturally-occurring amino acid of similar character, for example Gly<<→Ala, Val<→Ile<→Leu, Asp<→Glu, Lys<→Arg, Asn<→Gln or Phe<→Trp<→Tyr.

Substitutions encompassed may also be “non-conservative”, in which an amino acid residue which is present in a phospholipase inhibitory protein is substituted with an amino acid having different properties, such as a naturally-occurring amino acid from a different group (eg. substituted a charged or hydrophobic amino acid with alanine), or alternatively, in which a naturally-occurring amino acid is substituted with a non-conventional amino acid.

Amino acid substitutions are typically of single residues, but may be of multiple residues, either clustered or dispersed.

Amino add deletions will usually be of the order of about 1-10 amino acid residues, while insertions may be of any length. Deletions and insertions may be made to the N-terminus, the C-terminus or be internal deletions or insertions. Generally, insertions within the amino acid sequence will be smaller than amino- or carboxyl-terminal fusions and of the order of 1-4 amino acid residues.

In a preferred embodiment, neurotrophin-Trk signaling is up-regulated by contacting a cell with a peptide comprising, consisting essentially of, or consisting of an amino acid sequence selected from the group consisting of SEQ ID NO: 2, SEQ ID NO: 3, SEQ ID NO: 4, SEQ ID NO: 5, SEQ ID NO:7, SEQ ID NO: 8, SEQ ID NO: 9; SEQ ID NO: 10, and SEQ ID NO: 12. In another embodiment, the composition comprises the peptide of SEQ ID NO: 2.

The peptide may be fused to a carrier to facilitate transfer across the blood brain barrier. Accordingly, in one embodiment, the peptide selected from the group consisting of SEQ ID NO: 1, SEQ ID NO: 2, SEQ ID NO: 3, SEQ ID NO: 4, SEQ ID NO: 5 and SEQ ID NO: 6 comprises a carrier. In one embodiment, the carrier is the TAT amino acid sequence from HIV. In one embodiment, the TAT sequence is the amino acid sequence of YARAAARNARA (SEQ ID NO: 13). Accordingly, in one embodiment, the present disclosure provides a peptide comprising, consisting essentially of, or consisting of an amino acid sequence selected from the group consisting of SEQ ID NO:7, SEQ ID NO: 8, SEQ ID NO: 9; SEQ ID NO: 10, SEQ ID NO: 11 and SEQ ID NO: 12. Variants of the TAT sequence are known and may also be employed in the present disclosure.

Conversely, neurotrophin-Trk signaling may be directly down-regulated by contacting a cell with a compound that disrupts the binding of the p75^{ICD} juxtamembrane region to Trk. In one embodiment, the compound that disrupts the binding of the p75^{ICD} juxtamembrane region to Trk is an antibody that binds specifically to the p75^{ICD} juxtamembrane region SEQ ID NO: 1 or an antibody that binds specifically to the region of Trk that binds the p75^{ICD} juxtamembrane region.

Reference herein to antibody or antibodies includes whole polyclonal and monoclonal antibodies, and parts thereof, either alone or conjugated with other moieties. Antibody parts include Fab and F(ab)₂ fragments and single chain antibodies. The antibodies may be made in vivo in suitable laboratory animals, or, in the case of engineered antibodies (Single Chain Antibodies or SCABS, etc) using recombinant DNA techniques in vitro.

Means for preparing and characterizing antibodies are well known in the art (see, e.g., Antibodies: A Laboratory Manual, Cold Spring Harbor Laboratory, 1988, incorporated herein by reference). Conveniently, the antibodies may be prepared against a synthetic peptide based on the protein or peptide encoded by genes such as p75^{NTF} or Trk, for example p75^{ICD} juxtamembrane region SEQ ID NO: 1 or a peptide selected from of SEQ ID NO: 2, SEQ ID NO: 3, SEQ ID NO: 4. SEQ ID NO: 5 and SEQ ID NO: 6, or the region of TrkB that binds the p75^{ICD} juxtamembrane region SEQ ID NO: 1.

The antibodies described herein generally bind specifically to their respective targets. The phrase “binds specifically” to a polypeptide means that the binding of the antibody to the proteins of the invention is determinative of the presence of the proteins, in a heterogeneous population of proteins. Thus, the specified antibodies preferably bind to a particular protein at least two times the background and more typically more than 10 to 100 times background. Typically, antibodies of the invention bind to a protein of interest with a Kd of at least about 0.1 mM, more usually at least about 1 μM, preferably at least about 0.1 μM, and most preferably at least, 0.01 μM.

In another embodiment, the compound that disrupts the binding of the p75^{ICD} juxtamembrane region to Trk is a peptide that binds to Trk without enhancing signaling. Binding of such a peptide does not enhance signaling, and blocks the binding of p75^{ICD} to the Trk. An example of such a peptide is c15.21 (SEQ ID NO: 6 or 11).

Alternatively, interaction between the p75^{ICD} juxtamembrane region and Trk may be indirectly modulated, for example, by increasing or decreasing the endogenous levels of p75^{ICD} juxtamembrane region by modulating an upstream pathway. For example, p75^{NTR} α- and γ-secretases process p75^{NTR} to provide the p75^{ICD} juxtamembrane region (see FIG. 1). Accordingly, by increasing or decreasing the activity by these secretases, the endogenous levels of p75^{ICD} juxtamembrane region are modulated and therefore neurotrophin-Trk signaling regulated.

In one embodiment, the endogenous levels of p75^{ICD} juxtamembrane region in a cell are increased by (i) increasing α-secretase action; (ii) increasing γ-secretase action; or (iii) a combination thereof. In another embodiment, the endogenous levels of p75^{ICD} juxtamembrane region are decreased by (i) decreasing α-secretase action; (ii) decreasing γ-secretase action; or (iii) a combination thereof. Any α-secretase or γ-secretase modulators known in the art may be used and are well known to those skilled in the art. Examples of suitable p75^{NTR} α-secretase and p75^{NTR} γ-secretase inhibitors are TAPI-1 (N—(R)-[2-(Hydroxyaminocarbonyl)methyl]-4-methylpentanoyl-L-naphthylalanyl-L-alanine, 2-aminoethyl Amide); TAPI-2 (N—(R)-(2-(Hydroxyaminocarbonyl)Methyl)-4-Methylpentanoyl-L-t-Butyl-Glycine-L-Alanine 2-Aminoethyl Amide; BML-PI135-0001, Enzo Life Sciences, Inc., New York, USA) and Compound E ((S,S)-2-[2-(3,5-Difluorophenyl)-acetylamino]-N-(1-methy-2-oxo-5-phenyl-2,3-dihydro-1H-benzo[e][1,4]diazepin-3-yl)-propionamide; 565790 γ-Secretase Inhibitor XXI, Calbiochem®, Merck KGaA, Darmstadt, Germany), respectively.

The present disclosure also relates to compositions that can be used to modulate neurotrophin-Trk signaling. For example, a peptide, polypeptide, protein, or antibody that modulates the interaction between the p75^{ICD} juxtamembrane region and Trk, as discussed supra. The composition may also be a p75^{NTR} α- or γ-secretase modulator, also as discussed supra. In one embodiment, the composition is a peptide comprising, consisting essentially of, or consisting of an amino acid sequence selected from the group consisting of SEQ ID NO: 2, SEQ ID NO: 3. SEQ ID NO: 4, SEQ ID NO: 5, SEQ ID NO: 6, SEQ ID NO:7, SEQ ID NO: 8, SEQ ID NO: 9; SEQ ID NO: 10, SEQ ID NO: 11 and SEQ ID NO: 12. In another embodiment, the composition comprises the peptide of SEQ ID NO: 2 or SEQ ID NO: 7.

It will be appreciated that the present disclosure also relates to assays that can be used for identifying compounds that modulate neurotrophin-Trk signaling. The compounds may be identified by contacting a neurotrophin-Trk signaling system with a candidate compound and measuring the level of neurotrophin-Trk signaling in the presence and absence of the candidate compound. For example, the neurotrophin-Trk signaling system may comprise cells that endogenously express a Trk which is activated by a neurotrophin and treating said cells with a neurotrophin and c29 to produce conditions that promote neurotrophin-Trk signaling. Alternatively, the neurotrophin-Trk signaling system may comprise cells that are transfected with a construct comprising a gene encoding a Trk which is activated by a neurotrophin. In one embodiment, the neurotrophin-Trk signaling system is a BDNF-TrkB signaling system, wherein said system comprises cells that endogenously express TrkB and treating said cells with BDNF and c29. In one embodiment, the cells that endogenously express TrkB are motor neuron cells. In another embodiment, the neurotrophin-Trk signaling system is a NGF-TrkA signaling system, wherein said system comprises cells that endogenously express TrkA and treating said cells with NGF and c29. In one embodiment, the cells that endogenously express TrkA are PC12 cells.

As used herein the term “contacting” includes direct and indirect contacting of the neurotrophin-Trk signaling system with a candidate compound. The contacting may be by any means known in the art, for example, by adding the neurotrophin-Trk signaling system to the candidate compound, or vice versa.

The term “measuring” as used herein includes any method of measuring the level of neurotrophin-Trk signaling and may include detecting an up-regulation or down-regulation of neurotrophin-Trk signaling. For example, if the candidate compound is affecting the level of p75^{ICD} juxtamembrane region could also be used to determine the level of neurotrophin-Trk signaling. Alternatively, the level of a protein down-stream of Trk activation could be used to determine the level of neurotrophin-Trk signaling. Suitable down-stream proteins useful in the determining Trk activation would be well known to those skilled in the art. For example, p75^{ICD} is known to activate ERK1/2 signalling in TrkB- as well as TrkA-expressing cells (Ceni et al., 2010; Kommaddi et al., 2011b).

There are a number of methods known in the art for measuring the relative amount of proteins. For example, immunoassays such as the various types of immunoblotting, enzyme linked immunosorbent assays (ELISAs) and radioimmunoassays (RIA) are well known in the art. Other techniques such as Western blotting, dot blotting, FACS analyses, and the like may also be used. In one embodiment, immunoblotting is used to measure the level of phosphorylated Erk1/2 and/or phosphorylated Akt in the presence and absence of the candidate compound and determine the level of NGF-TrkA signaling.

The difference in the level of neurotrophin-Trk signaling is preferably statistically significant i.e., greater than what might be expected to happen by chance alone. Significance is typically defined by an appropriately small p value, such as p<0.05.

The compounds described herein may be used to treat diseases and disorders related to neurotrophin-Trk signaling by, for example, administering to a subject in need thereof a therapeutically effective amount of a compound in order to regulate neurotrophin-Trk signaling.

Generally, the terms “treating,” “treatment” and the like are used herein to mean affecting a subject, e.g. human individual or animal, its tissue or cells to obtain a desired pharmacological and/or physiological effect. The effect may be prophylactic in terms of completely or partially preventing the condition or sign or symptom thereof, and/or may be therapeutic in terms of a partial or complete cure of the condition. “Treating” as used herein covers any treatment of, or prevention of a condition associated with or exacerbated by neurotrophin-Trk signaling in a vertebrate, a mammal, particularly a human, and includes: (a) preventing the condition from occurring in a subject that may be predisposed to the condition, but has not yet been diagnosed as having it; (b) inhibiting the condition, i.e., arresting its development; or (c) relieving or ameliorating the condition, i.e., cause regression of the symptoms.

The term “subject” as used herein refers to an animal subject in which the control of neurotrophin-Trk signaling is desirable. The subject may be a human, or may be a domestic, companion or zoo animal. While it is particularly contemplated that the compounds described herein will be suitable for use in the medical treatment of humans, they are also applicable to veterinary treatment, including treatment of companion animals such as dogs and cats, and domestic animals such as horses, cattle and sheep, or zoo animals such as non-human primates, felids, canids, bovids, and ungulates.

The process of memory acquisition and extinction, the latter of which assists in the removal of conditioned fear memory, is mediated by BDNF-TrkB signaling. Accordingly, a method of promoting memory extinction or treating a fear-related disorder could involve administering to the subject a therapeutically effective amount of a composition capable of up-regulating BDNF-TrkB signaling, such a composition comprising a polypeptide of SEQ ID NO: 2 or SEQ ID NO: 7.

Further, there are a number of psychiatric disorders that are known to involve a reduction in BDNF and/or NT3, such as in schizophrenia, depression and other mood disorders, such as bipolar spectrum disorder. Other disorders include anxiety, drug addiction, obsessive-compulsive disorder and Autism spectral disorder. Accordingly, in one embodiment, the disorder is a psychiatric disorder. In another embodiment, the psychiatric disorder is schizophrenia, depression, bipolar spectrum disorder, anxiety, drug addiction, obsessive-compulsive disorder and Autism spectral disorder.

Therefore, the treatment of these disorders may also involve administering to a subject a therapeutically effective amount of a composition capable of up-regulating neurotrophin-Trk signaling. In one embodiment, the composition capable of up-regulating neurotrophin-Trk signaling comprises a peptide comprising, consisting essentially of, or consisting of the amino acid sequence selected from the group consisting of SEQ ID NO: 2, SEQ ID NO: 3, SEQ ID NO: 4, SEQ ID NO: 5, SEQ ID NO: 7, SEQ ID NO: 8, SEQ ID NO: 9, SEQ ID NO: 10 and SEQ ID NO: 12. In another embodiment, the composition capable of up-regulating BDNF-TrkB signaling comprises a polypeptide of SEQ ID NO: 2 or 7.

Neurotrophin-Trk signaling promotes neuritre outgrowth. Therefore, a method of promoting neuritre outgrowth in a subject in need thereof may comprise administering to a subject a therapeutically effective amount of a composition capable of up-regulating Neurotrophin-Trk signaling. The promotion of neuritre outgrowth could be used to treat neurodegenerative injuries and disorders, such as cerebral palsy, trauma induced paralysis, vascular ischaemia associated with stroke, neural tumours, motoneurone disease, Parkinson's disease, Huntington's disease, Alzheimer's disease, multiple sclerosis and peripheral neuropathies associated with diabetes, heavy metal or alcohol toxicity, renal failure and/or infectious diseases such as herpes, rubella, measles, chicken pox, HIV and/or HTLV-1.

Due to the role of neurotrophin-Trk signaling on neurons, compositions capable of up-regulating neurotrophin-Trk signaling could also be considered cognitive enhancers. In one embodiment, the cognitive enhancer comprises a peptide, polypeptide or protein that corresponds to all or part of the p75^{ICD} juxtamembrane region SEQ ID NO: 1. In another embodiment, the cognitive enhancer is a peptide comprising, consisting essentially of, or consisting of an amino acid sequence selected from the group consisting of SEQ ID NO: 2, SEQ ID NO: 3, SEQ ID NO: 4, SEQ ID NO: 5, SEQ ID NO: 6, SEQ ID NO:7, SEQ ID NO: 8, SEQ ID NO: 9; SEQ ID NO: 10, SEQ ID NO: 11 and SEQ ID NO: 12.

The compounds disclosed herein may also be used to treat diseases or disorder exacerbated by neurotrophin-Trk signaling. For example, BDNF-TrkB signaling has recently been linked to chemotherapy resistance in Head and Neck Squamous Cell Carcinoma. In this case the treatment of such diseases or disorders would involve administering a therapeutically effective amount of a composition comprising a compound capable of down-regulating neurotrophin-Trk signaling. In another example, NGF-TrkA signalling can contribute to chronic pain. Thus, treatment of chronic pain would involve administration of a therapeutically effective amount of a composition comprising a compound capable of down-regulating neurotrophin-Trk signaling. Examples of such compounds include antibodies that bind specifically to the p75^{ICD} juxtamembrane region or the region of Trk that binds the p75^{ICD} juxtamembrane region. Other examples include p75^{NTR} α-secretase and p75^{NTR} γ-secretase inhibitors, or peptides which bind Trk without signaling and block interaction between p75ICD and Trk such as SEQ ID NO: 6 or 11.

It will be appreciated that the present disclosure also relates to methods of diagnosing diseases or disorders mediated by neurotrophin-Trk signaling.

The disclosure will now be further described by way of reference only to the following non-limiting examples. It should be understood, however, that the examples following are illustrative only, and should not be taken in any way as a restriction on the generality of the invention described above.

### EXAMPLES

### Example 1

### Antibodies and Growth Factors

The following antibodies were used for Western blotting: rabbit anti-p75^{NTR} (1:2000; Promega anti-p75^{ICD}, 1:1000 Abcam anti-p75ICD), rabbit anti-TrkA 1:500 (Upstate), mouse anti-phoeERK1/2 1:1000, and rabbit anti-panERK 1:1000 (Millipore), rabbit anti-phosAkt 1:2000, mouse anti-panAkt 1:2000 (Cell Signaling); rabbit anti-GFP 1:1000, rabbit anti-p75NTR (Roche); donkey anti-rabbit 680 secondary (1:10000) and donkey anti-mouse 800 secondary 1:50000 (Invitrogen). For live cell immunostaining, rabbit anti-TrkA extracellular domain agonist antibody (1:500), was kindly provided by Louis Reichardt (University of California, San Francisco); monoclonal antibody against rat p75^{NTR} extracellular domain (MC192) was purified from hybridoma conditioned supernatant and conjugated to FITC (Sigma-Aldrich) in our lab. Mouse monoclonal antibody against murine p75^{NTR} extracellular domain (clone MLR-2) and rabbit polyclonal anti-TrkB antibody were a kind gift from Robert Rush (Flinders University). The following growth factors were used: purified mouse nerve growth factor (NGF) (Biosensis), recombinant human BDNF (Millipore), recombinant rat-CNTF (R&D Systems) and recombinant human GDNF (Peprotech).

### Example 2

### Expression Constructs

The p75^{NTR} constructs p75^{NGLY}, p75^{ICD} and p75^{CTF} (Underwood et al., 2008), and p75^{Δ-JUX} (Coulson et al., 2000) have been described previously. The TrkA and TrkAK/N constructs are described in (Underwood et al., 2008). p75^{NTR}-YFP expression constructs used a modified pCDNA3 (Invitrogen) backbone. The rat p75^{NTR} signal peptide including a Kozak sequence (nucleotides −29-87) was inserted between the KpnI and EcoRV restriction sites, generating the vector pCDNA3-SP. The fluorophore enhanced YFP was amplified by polymerase chain reaction (PCR) from peYFP-N1 (Clontech), using primers incorporating a 5′EcoRV and NheI restriction sites and a 3′ primer incorporating a stop codon and XhoI. YFP was cloned in frame between the EcoRV and XhoI restriction sites of pCDNA3-SP, generating the vector and pCDNA3-YFP. p75^{NTR} mature peptide coding sequences were amplified under standard PCR conditions with 5′ EcoRV and 3′ NheI restriction sites incorporated into the respective primers. Subsequently, p75^{NTR} coding sequences were cloned Matusica et al. Supplemental Information (compiled Word file) between EcoRV and NheI restriction sites of pCDNA3-YFP vector to generate inframe carboxyl-tagged fusion proteins. The EGFR-GFP construct was generously provided by Rob Parton (The University of Queensland).

### Example 3

### c29 and Control Peptide Synthesis

29 amino acid residue peptide of the juxtamembrane ‘Chopper’ domain (Coulson et al., 2000) (c29: KRWNSCKQNKQGANSRPVNQTPPPEGEKL) and a randomly scrambled version (SC: SKGQVCRNQPGQNKPEPANKSWKETPLRN) were synthesized either as N-terminal fusions to a non-naturally occurring protein transduction domain (PTD4) peptide (PTD4: YARAAARNARA; (Ho et al., 2001) using t-boc chemistry and purified using reverse phase HPLC by Dr. James I. Elliott at Yale University (New Haven, Conn.), or as disulfide-liked peptides (Coulson et al., 2000) by Auspep Pty. Ltd (Tullamarine, Australia). No difference in functionality was seen between the two conjugation methods, and no effects were seen in cells treated with PTD4 alone, peptides without carrier or disulfide-liked peptides that had been reduced with DTT. For immunoprecipitation experiments, the c29 peptide was labelled on the amino terminus with biotin via a six-carbon spacer. The biotinylated control peptide mimicking the p75^{NTR} extracellular juxtamembrane domain LC1 (HRGTTDNLIGGSC-NH2) was manufactured by (Auspep).

### Example 4

### P12 Cell Culture and Transient Transfection

PC12 were cultured as described previously (Greene and Tischler, 1976). In experiments assessing the functions of various p75^{NTR} constructs, PC12 cells were electroporated using the Neon Transfection System with one pulse of 1410 volts and a width of 30 ms. Cells were then plated in 12 well culture plates coated with poly-Lornithine (0.015%) and 2 μg/ml of murine laminin, and treatments were applied as described. For detailed construct information see Example 2.

### Example 5

### PC12 Cell Neurite Outgrowth, Viability and NGF Withdrawal Assays

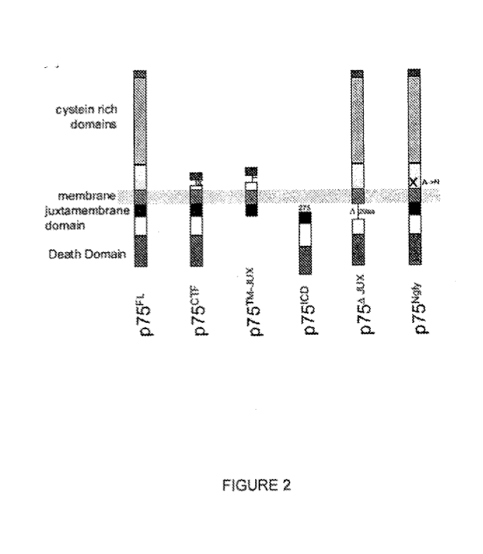

To assess the effect of p75^{NTR} constructs (FIG. 2) or c29 peptide on TrkA-mediated neuritogenesis, transfected PC12 cells were grown in the presence of 100 ng/ml NGF for 3 days, with fresh medium and NGF treatments added on day 2. Cells were then imaged live on an Olympus (IX81) microscope fitted with a CO2 atmospheric chamber, using analySIS software. Four random images of 80-100 cells were taken for each treatment condition and images were determined by measuring polygon neurite length of all cells in each image. Data analysis was performed using Graphpad PRISM.

To investigate the role of c29 peptide in NGF-withdrawal assays, NGF-containing medium in PC12 cultures was exchanged for NGF-free medium on day 3, and cells were treated with c29 or scrambled peptide and reduced NGF concentrations (1 ng or 10 ng/ml). Treated cells were cultured for 2 days and cell viability was determined via quantification of cytosolic acid phosphatase enzyme activity as described in (Yang et al., 1996).

Briefly, cells were grown in 96-well plates at densities of 5000 cells per well. The culture medium in treated cell assays was removed from and each well was washed once with 200 μl phosphate buffered saline (PBS, pH 7.2). To each well, 100 μl of sodium acetate and p-nitrophenyl phosphate buffer containing 0.1 M sodium acetate (pH 5.0), 0.1% Triton X-100, and 5 mM p-nitrophenyl phosphate was added. The plates were placed in a 37° C. incubator for 30 minutes. The reaction was stopped with the addition of 10 ml of 1 N NaOH, and colour development absorbance was assayed at 405 nm using a microplate reader (POLARstar OPTIMA, USA). The non-enzymatic hydrolysis of the pap substrate was determined for each assay by including wells that did not contain cells. This background value was typically 0.03-0.1 absorbance units.

### Example 6

### HEK293 Cell Culture and Transient Transfections

HEK293 fibroblast cells (transformed by sheared adenovirus type 5 DNA-HEK293AD cells) were cultured as described in (Shaw et al., 2002). HEK293 cells were transfected with Fugene 6 (Roche) as per the manufacturer's instructions, harvested 48 hours later, and were used for flow cytometry experiments or lysed for immunoblotting.

### Example 7

### Primary Motor Neuron Culture, Survival and Growth Factor Withdrawal Assays

Cultures of spinal motor neurons prepared from embryonic day 13 C57BL6J mice were isolated by a method previously described by (Wiese et al., 2010). Briefly, pregnant mothers were sacrificed by cervical dislocation and the uterus removed by Caesarean section and placed in Neurobasal medium. Lumbar spinal cords were dissected from E13 embryos on ice. Batches of 2 or 3 spinal cords were incubated in 1 mL 0.05% trypsin-ETDA for 10 minutes at 37° C. in 15 mL tubes. To prevent further trypsinisation, 0.14 mg/mL trypsin inhibitor solution was added and the tissue centrifuged at 100×g for 7 minutes, without acceleration or deceleration. The supernatant was removed and the spinal cord tissue was resuspended in 1 mL of Neurobasal Medium (NB). In order to obtain a single cell suspension, each batch of 2-3 spinal cords was agitated manually for 2 minutes. The suspension was allowed to settle and the supernatant collected after being filtered through a 100 μm sieve. The remaining tissue was further triturated ˜8 times through a P200 pipette and filtered through a 100 μm sieve. The combined supernatants were then separated by centrifugation on a 5 ml cushion of 9% (vol/vol) OptiPrep® gradient in NB containing 2% B27 supplement for 45 min at 850×g, without acceleration or deceleration. To prevent toxicity following Optiprep® separation, the layer containing MNs was harvested into 10 mL of NB containing 10% HS, and the cell suspension was centrifuged at 900×g for 7 minutes at 24° C., without acceleration or deceleration. The supernatant was removed and the cell pellet was resuspended in 1 mL of NB medium.

The number of viable motor neurons was counted on a haemocytometer using 0.08% trypan blue and neurons plated in 4-well culture dishes at a density of 3,000 cells per well in 100 μL NB containing 10% HS. Prior to plating, the culture dishes were precoated with 0.0015% poly-ornithine overnight at 4° C., washed 3 times with NB medium, then coated with 3.75 μg/mL laminin for 30 minutes at 37° C. and washed 3 times with NB medium. Cells were allowed to adhere for 1 hour at 37° C. and then 2 mL of NB supplemented with 1% B27, 500 μM Glutamax, 10% HS, 25 μM β-mercaptoethanol and 1 ng/mL BDNF, CNTF and/or GDNF was added. Primary motor neurons were maintained at 37° C. in a humidified atmosphere with 5% CO2 and culture medium was replaced at day 1 and 3. Phase contrast images of motor neurons were taken using an Olympus (6IX81) microscope after motor neurons were grown in vitro for up to 7 days.

To assess the effects of c29 peptide on motor neuronal survival in limiting growth factor concentrations, isolated spinal motor neurons were plated and treated with 0.01, 0.1 or 1.0 ng/ml of BDNF in the presence of 1 μm PTD4-linked c29 or scrambled peptide, and cultured for 5 days with the addition of fresh BDNF and peptide treatments on day 2 and 4. For growth factor-withdrawal assays, following initial plating in 1 ng/ml of BDNF, CNTF or GDNF, cultures were washed four times with serum-free Neurobasal (NB) medium, after which the medium was replaced with medium containing 1-0.01 ng/ml of BDNF, CNTF or GDNF and 1 μM PTD4-linked c29 or scrambled peptides, as indicated. For assays investigating the effect of c29 on NGF-mediated motor neuron death, cultures were grown in 1 ng/ml BDNF, CNTF or GDNF for 1 day, before being treated with 100 ng/ml 2.5S NGF in and 1 μM PTD4-linked c29 or scrambled peptide, as indicated. Motor neurons were counted on day 1, and to assess the effects of various treatments days 3 and 5 (4 separate 10×10 grid fields of each well per experiment). The Animal Ethics Committee of the University of Queensland approved experiments involving the use of animals, which were used in accordance with the Australian Code of Practice for the Care and Use of Animals for Scientific Purposes.

### Example 8

### Cell Lysis, Immunoprecipitation and Immunoblotting

In biochemical experiments, PC12 and HEK293 cells were lysed for 20 minutes on ice in lysis buffer as described by (Ceni et al., 2010). Briefly, the lysis buffer contained 10 mM Tris-HCl, 150 mM NaCl, 2 mM EDTA, 1% (v/v) NP-40, 1% (v/v) TX-100, 10% Glycerol, 1 mM PMSF, 1 mM Sodium Orthovanadate, 1 μM BB94, 1% (v/v) Roche protease inhibitor cocktail and pH 8.0 (Ceni et al., 2010).

For peptide pull down assays, the biotinylated c29 or LC1 control peptides were incubated with protein A-sepharose beads or Dynabeads MyOne Streptavidin T1 (Invitrogen) overnight at 4° C. Cell lysates were then added to the peptide-protein A-sepharose complex or Dynabeads MyOne Streptavidin T1 and incubated for 3 hours at 4° C. Peptide-receptor complexes were washed extensively with RIPA lysis buffer and eluted by boiling in Laemmli sample buffer. For p75^{NTR} construct immunoprecipitations, HEK293 cells were treated with NGF at 50 ng/ml for 10 minutes 48 hours after transfection with 1 μg DNA per well. Cells were immediately harvested in ice-cold phosphate buffered saline (PBS) and washed twice, then lysed in 1 ml of lysis buffer. Cell lysates were pre-cleared in 75 μl (1:2) protein-G sepharose: PBS by rotation end-over-end for 2 hours at 4′C. Lysates were then incubated with 3-5 μg antibody, either mouse-anti-YFP/GFP (Roche) or rabbit-anti-human p75^{NTR} intracellular domain antibody (Promega), and rotated end-over-end for 2 hours at 4° C.

Immunoprecipitation was performed by the addition of 75 μl (1:2) protein-G sepharose: PBS and incubation end-over-end for 16 hours at 4° C. The immunoprecipitate was washed 4 times with 1 ml of PBS then eluted in 60 μl 2×LDS sample buffer. For immunoblotting, samples were boiled for 5 minutes, separated by SDS-PAGE and transferred onto nitrocellulose membranes, then western blotted using standard protocols (see Example 1 for antibodies used).

Immunoreactive bands were detected using secondary antibodies labeled with 680 nm and 800 nm emitting fluorophores (LICOR).

### Example 9

### Motor Neuron Axotomy

Median and ulnar nerve axotomy was performed on 4 day-old Wistar rat pups as previously described (Cheema et al., 1996). Peptides (c29 and vehicle only; 100 nM) were applied via a 1 mm³ cube of pluronic gel foam that was sutured to the proximal axonal stump (Cheema et al., 1996). After 5 days the animals were perfused with a 4% solution of paraformaldehyde in phosphate buffer. Cervical spinal cords were removed, after which serial sections were cut at 40 μm, mounted onto gelatin-coated slides and stained in 0.1% cresyl violet. Details of the counting procedure have been described previously (Cheema et al., 1994).

### Example 10

### NGF Binding and Binding Competition Assays

The NGF was conjugated to FITC by standard methods and detailed flow cytometry acquisition and analysis. For NGF-FITC conjugation 400 μg βNGF in 200 μl pH 8.5 NaHCO3 buffer was mixed with 5-10 μl of 10 mg/ml FITC in 100 μl DMSO for 10 hours at 4° C. on an orbital shaker set to 100 rpm. The excess FITC was removed by dialysis against 3 changes of 5 L of 10 mM Tris, 150 mM NaCl, 0.2% acetic acid, pH 8.2 for 24 hours at 4° C. Conjugate F/P characterization was determined by measuring absorbance at 280 nm and 495 nm according to manufacturer's instructions. Biological activity of the NGF-FITC conjugate was tested by performing PC12 cell neurite outgrowth assays unlabelled NGF, and comparing the levels of neuritogenesis. NGF-FITC conjugates were used within 4 weeks of synthesis.

For the flow cytometry NGF binding assays, fluorescence cell parameters of at least 20,000 events for PC12 cells and 100,000 events for HEK293 cells were acquired by flow cytometry list mode and measurements were performed on a single cell basis (with compensation for double event counting). Dead cells and debris were gated out of the analysis on the basis of forward scatter fluorescence, and mean, median and maximal fluorescence values of the gated cell populations were analysed using FACS Diva Software (Becton-Dickinson Biosciences) and FACS Express Software (Becton-Dickinson Biosciences).

Background fluorescence of HEK293 cells was non-transfected cells (±c29 or scrambled peptide treatment) treated with 26 nM NGF-FITC. For PC12 cells, the control population was cells that had not been treated with NGF-FITC. Analysis of real-time NGF-FITC receptor binding experiments was performed on 550,000-600,000 cells at 4° C. in serum free NB medium and binding kinetics were calculated from raw data files compressed to 50,000 binding events using FlowJo and MatLab Software. Data was plotted with the use of GraphPad Prism.

Only NGF-FITC preparations that promoted equivalent neurite outgrowth of PC12 cells to that of unlabelled NGF were used in binding experiments. To investigate NGF binding, PC12 or transfected HEK293 cells were serum starved for 4 hours, harvested, washed with Dulbecco's Modified Eagle Medium (DMEM), and counted. When used, c29 or scrambled peptide were added to cultures for the third hour of the serum starvation procedure. 1×10⁷ cells/ml suspended in 500 μl of serum-free DMEM were incubated with 26 nM NGF-FITC on ice for 60 minutes before analysis. For binding competition assays, following 1 hour in NGF-FITC, cells were washed with ice-cold PBS containing 0.1% normal bovine serum and incubated on ice with unlabeled NGF at concentrations ranging from 100 pM to 10 μM for 30 minutes, then washed twice in PBS before being analysed on a BD Flow Cytometer FACS Scan (Becton-Dickinson).

To determine association rates, cells were harvested as above following serum starvation and peptide treatment, and were resuspended in 3 ml of serum-free Neurobasal (NB) medium containing 2 nM glutamax (Invitrogen) and 1 nM nonessential amino acids, prior to analysis on the flow cytometer. Cells were analysed for 30 seconds prior to the addition of 26 nM NGF-FITC, and then for 30 minutes in real time with an analysis rate of 200-300 events/sec.

### Example 11

### p75^{CTF} Promotes Cell Death Whereas p75^{ICD} Promotes Neuritre Outgrowth in PC12 Cells

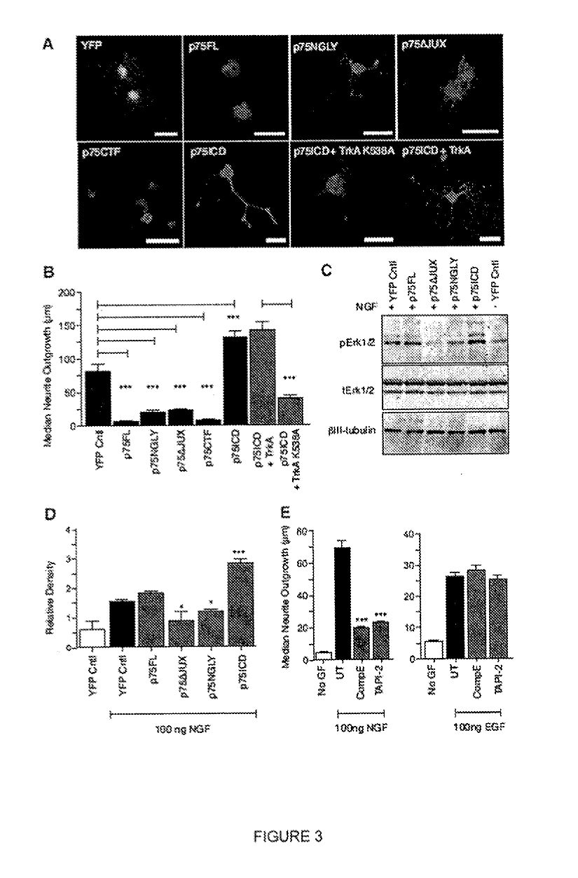

To determine the role of the different cleavage fragments of p75^{NTR} in the context of TrkA activation, yellow fluorescent protein (YFP)-tagged p75^{NTR} constructs (FIG. 2) were transiently overexpressed in PC12 cells, a neuronal model widely used for studying the actions of neurotrophins as the cells express endogenous TrkA and p75^{NTR}. Cultures were then treated with 100 ng/ml NGF for 5 days to induce differentiation and promote neurite outgrowth, responses which are mediated by TrkA signals (Green et al., 1986; Inagaki et al., 1995; Vaudry et al., 2002). As previously reported (Barrett and Georgiou, 1996), overexpression of a full-length p75^{NTR} construct (p75^{FL}) led to large-scale cell death, with the few remaining transfected cells having only neurite stumps (FIG. 3A,B). PC12 cell overexpression of a control YFP-only plasmid did not result in overt toxicity (FIG. 3A). Similarly, the expression of a p75^{NTR} construct that does not undergo cleavage due to the inclusion of an N-glycosylation mutation at the α-secretase cleavage site, p75^{NGLY} (Underwood et al., 2008) had no discernable effect on cell survival, indicating that a cleavage fragment of p75^{NTR} is required to promote cell death. However, the neurite outgrowth of the p75^{NGLY}-expressing PC12 cells was stunted (length less than the diameter of cell body) compared to that of control YFP-transfected cells (FIG. 3A, B). Cells expressing a construct containing a 33 amino acid deletion of the intracellular juxtamembrane region (p75^{Δ-JUX}) remained viable in culture (FIG. 3A), consistent with a pro-apoptotic role of the membrane-tethered “Chopper” domain reported in our previous studies (Coulson et al., 2000; Coulson et al., 1999a). However the neurites of the p75^{Δ-JUX}-expressing cells were also shorter than those of control YFP-expressing cells (FIG. 3A, B). PC12 cells overexpressing a construct mimicking p75^{CTF}, and which does not undergo γ-secretase cleavage (Underwood et al., 2008), did not typically survive in culture past 48 hours, and the expression of the YFP reporter observed at 5 days was predominantly associated with cell debris (FIG. 3A), thereby confirming that p75^{CTF} is a potent cell death signalling protein in PC12 cells, as previously reported (Coulson et al., 2000). By contrast, overexpression of the p75^{ICD} construct resulted in robust neurite outgrowth, with neurites 60% longer than those observed in control YFP-expressing cells (FIG. 3A, B). These findings support the idea that cleavage of p75^{NTR} is a key regulator of its function, with p75^{CTF} inducing cell death and p75^{ICD} potentiating neurite outgrowth.

As NGF-mediated PC12 cell differentiation is known to occur via TrkA activation (Chao and Hempstead, 1995; Green et al., 1986), we next tested whether TrkA activity was required for p75^{ICD} overexpression to potentiate neurite outgrowth in PC12 cells. To achieve this we expressed a TrkA receptor with a tyrosine point mutation in the first phosphorylation loop (TrkAK538A), which cannot autophosphorylate, and thus acts as a dominant-negative receptor (Yano et al., 2001). When p75^{ICD} was co-expressed with TrkAK538A, neurite outgrowth was almost totally abrogated, with only 10-40 μm neurites observed following NGF treatment (FIG. 3A,B). Overexpression of a functional TrkA construct together with p75^{ICD} again resulted in robust differentiation (FIG. 3A, B). This indicated that p75^{ICD} promotes TrkA-dependent signals, resulting in PC12 cell differentiation and neurite outgrowth.

NGF-induced neurite outgrowth in PC12 cells depends on increased and sustained activation of extracellular signal-regulated kinase Erk1/2 by TrkA (Kaplan and Stephens, 1994; Qui and Green, 1992; Vaudry et al., 2002). To measure the effect of p75^{NTR} and its cleaved moieties on TrkA activation, we assessed the levels of phosphorylated Erk1/2 (pErk1/2) in PC12 cells expressing various p75^{NTR} constructs 24 hours after NGF treatment. Immunoblot analysis of pErk1/2 revealed increased Erk1/2 activation in cells expressing p75ICD (FIG. 3C, D). Cells transfected with full-length p75^{NTR} also showed increased pErk1/2 activity, whereas those expressing the non-cleavable p75^{NGLY} or p75^{Δ-JUX} constructs showed reduced pErk1/2 signalling, with levels of activated Erk1/2 equivalent to or less than that of cells not exposed to NGF stimulation (FIG. 3C, D).

As PC12 cells express endogenous p75^{NTR}, we next tested whether the endogenous production of p75^{ICD} is critical for PC12 cell differentiation by using inhibitors of the p75^{NTR} α- and γ-secretases, TAPI-2 and compound E, respectively (Underwood et al., 2008). Cells were treated with NGF and α- and γ-secretase inhibitors over 5 days, after which the length of their neurites was measured. Cells in both α- and γ-secretase inhibitor-treated cultures had significantly shorter neurites (20 μm) than those of control (NGF-treated) cells (FIG. 3E). In contrast, neither the α- nor the γ-secretase inhibitor had any significant effect on PC12 cell differentiation induced by epidermal growth factor (EGF), which has a limited ability to differentiate PC12 cells at high concentrations and activates Erk1/2 through a related tyrosine kinase EGF receptor (Traverse et al., 1992) (FIG. 3E).

These data indicate that each of the inhibitors was acting on a substrate(s) involved specifically in NGF-induced differentiation through TrkA. As cleavage of p75^{NTR} is known to be altered by both TAPI-2 and compound E, this result is consistent with the idea that p75^{NTR} proteolysis, thereby generating p75^{ICD}, is required for robust TrkA-mediated neurite outgrowth.

### Example 12

### TrkA Interacts with the Intracellular Juxtamembrane Domain of p75NTR

As TrkA and p75^{NTR} have previously been shown to interact (Huber and Chao, 1995; Jing et al., 1992; Wolf et al., 1995), we then investigated whether a cleaved p75^{NTR}/TrkA complex is retained following p75^{NTR} proteolysis. TrkA-p75^{NTR} interactions were examined via immunoprecipitation following transfection of HEK293 cells.

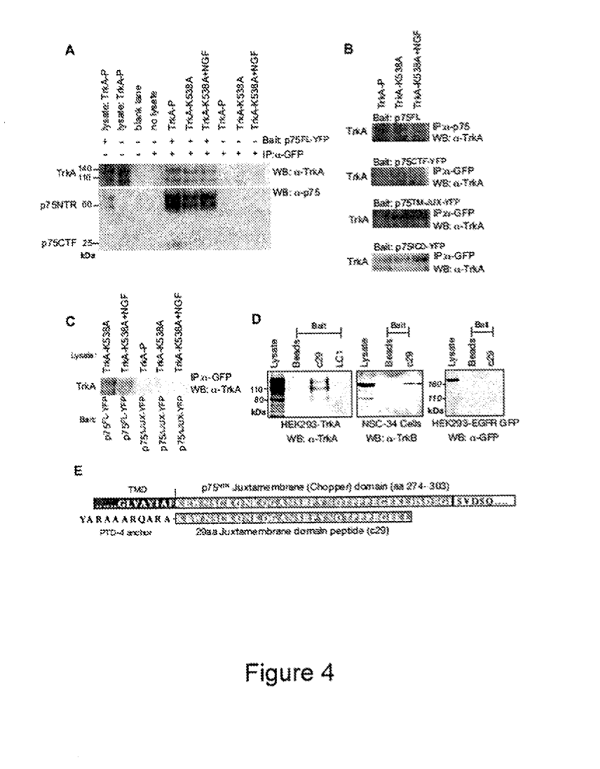

Full-length and truncated p75^{NTR} constructs were transfected together with either kinase-active TrkA or kinase-inactive TrkAK538A. The ability of p75^{NTR} to associate with TrkA in the presence or absence of NGF was also determined. Both full-length p75^{NTR} and its C-terminal fragment were able to co-immunoprecipitate TrkA and TrkAK538A (FIG. 4A, B), indicating that the interaction was not mediated via the extracellular domain of p75^{NTR}. Consistent with this, neither TrkA activation nor dimerisation by NGF significantly influenced the amount of TrkA that was coimmunoprecipitated with p75^{NTR} (FIG. 4A, B). p75^{ICD} was also able to pull down TrkA (FIG. 4B).

We then further mapped the domain of p75^{NTR} required for the interaction, revealing that the truncated p75^{TM-JUX} protein (consisting of only the transmembrane and 35 amino acid intracellular juxtamembrane membrane domains of p75^{NTR}) immunoprecipitated TrkA, whereas the p75^{Δ-JUX} protein (expressed by the construct lacking 33 amino acids of the Intracellular juxtamembrane region) did not pull down TrkA (FIG. 4C). In addition, a synthetic 29 amino acid juxtamembrane domain peptide (c29) (FIG. 4E), but not a scrambled 29 amino acid control peptide (SC), was also able to pull down TrkA (FIG. 4D).

Consistent with the ability of p75^{NTR} to interact with the other members of the Trk family (Bibel et al., 1999, Vesa et al, 2000, Hartmann et al. 2004), c29 was also able to pull down endogenously expressed TrkB from the motor neuron-like cell line NSC-34 (FIG. 4D), interacting, notably, with the truncated form of TrkB which is the major form of TrkB expressed in these cells (Matusica et al., 2008). However, c29 did not pull down the tyrosine kinase-related EGF receptor (FIG. 4D).

These observations define the juxtamembrane “Chopper” region of p75^{NTR} as necessary and sufficient for interaction with TrkA and TrkB, but also indicate that the interaction does not extend to a tyrosine kinase receptor outside the Trk family.

### Example 13

### The c29 Peptide Enhances NGF-Mediated Differentiation and Survival

To test the ability of c29 to enhance Trk function we first used two experimental PC12 cell paradigms: differentiation and serum withdrawal. Although the amount of NGF reportedly required to differentiate PC12 cells usually varies between 50-100 ng/ml in the literature, lower NGF concentrations have in some cases been reported to stimulate this process. Thus, exposure to a lower amount of NGF has been reported to “prime” PC12 cells, making them more sensitive to a subsequent low growth factor application, which then results in differentiation (Greene et al., 1975).

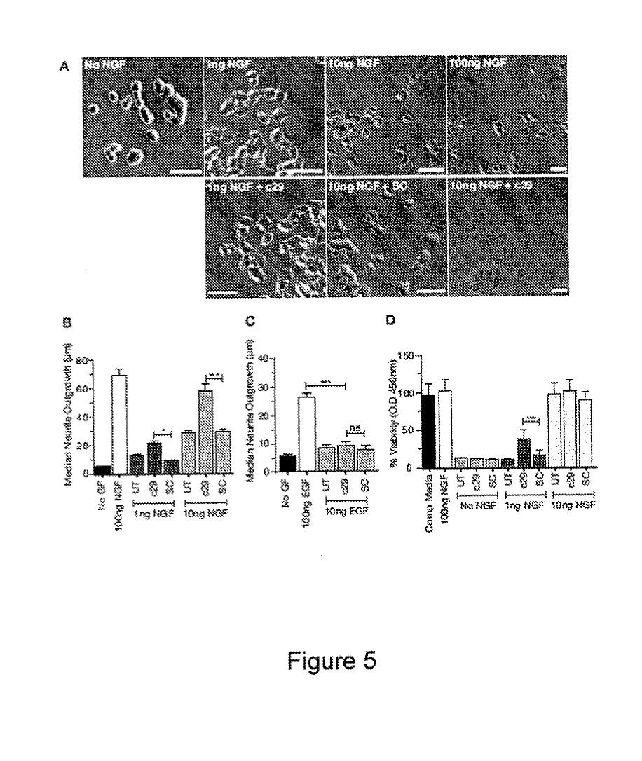

Once PC12 cells are fully differentiated, they become dependent on NGF for survival (Greene, 1978), but can survive if grown in the presence of lower concentrations of NGF (Greene and Tischler, 1976). To determine if c29 could potentiate TrkA-mediated signals, PC12 cells were cultured in the presence of NGF concentrations ranging from 1-100 ng/ml for 5 days, following serum starvation and pre-incubation with c29, scrambled control peptide or no peptide (FIG. 5A). Rapid intracellular delivery of c29 or the scrambled control peptide was achieved by linking the peptides to a 10-residue cell-permeable protein transduction domain peptide (PTD4), possessing enhanced protein transduction potential (Ho et al., 2001). Peptides (1 μM) were added to the cultures every 48 hours at the time of NGF addition and medium exchange. Day 5 analysis of the median neurite outgrowth of cultures grown in 100 ng/ml NGF revealed no difference between the c29-treated, scrambled peptide-treated and untreated cultures, as all cells had extensive neurite growth (FIG. 5A, B). However, cells treated with lower concentrations of NGF (1 ng/ml and 10 ng/ml) and grown in the presence of c29 produced significantly longer neurites than cultures treated with the scrambled peptide or NGF alone (FIG. 5B).

Indeed, cells treated with c29 and 10 ng/ml NGF had a median neurite length of 60 μm, comparable with that of control 100 ng/ml NGF-treated cells, whereas the control conditions produced neurites of 35 μm (FIG. 5A, B). Similarly, the effect of c29 on PC12 cells cultured in 1 ng/ml NGF also revealed a significant enhancement of neurite outgrowth to 20 μm, which was comparable to the neurite length of cells in control 10 ng/ml NGF-treated cultures (FIG. 5A, B).

To determine whether the effect of the c29 peptide on enhancement of neurite outgrowth was NGF and TrkA specific, we repeated the experiment using EGF to stimulate differentiation. In EGF-treated cultures c29 had no effect on neuritogenesis when compared to no peptide- or scrambled peptide-treated cultures (FIG. 5C).

We next investigated whether c29 could affect other cellular functions. Consistent with c29 requiring TrkA activity to exert its actions, c29 had no effect on basal PC12 cell growth and proliferation in medium lacking NGF (not shown). However, analysis of survival rates of PC12 cell cultures, following withdrawal or reduction of NGF concentrations after differentiation in 100 ng/ml NGF, revealed that c29 significantly increased the survival rate of cells in the presence of 1 ng/ml NGF (FIG. 5D). Under control conditions, ing/ml NGF was not sufficient to maintain viable PC12 cell cultures, whereas 10 ng/ml NGF ensured maximal survival (FIG. 5D). Importantly, c29 did not affect on cell survival in the absence of NGF (FIG. 3D), confirming that c29 has no significant effect on its own. This observation is indicative of a synergistic action of c29 with activated TrkA.

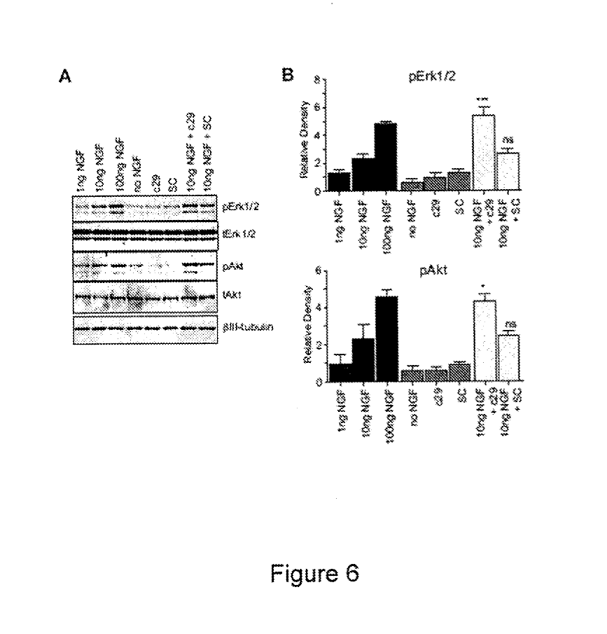

The observed functional effects on neurite outgrowth and survival were next correlated with enhanced activation of signal transduction pathways that mediate the trophic effects of TrkA. Lysates of cells treated for 24 hours with NGF alone or in combination with c29 or scrambled peptide were immunoblotted for pErk1/2 (neurite outgrowth), and the serine/threonine protein kinase Akt (survival). NGF treatment upregulated the level of pErk1/2 and phosphorylated Akt (pAkt), in a dose-dependent fashion (FIG. 46A, B). Total levels of Erk1/2 or Akt were unaffected by any treatment (FIG. 6 A, B).

In the absence of NGF, treatment of cells with c29 or scrambled peptide had no effect on pErk1/2 or pAkt signalling (FIG. 6A, B). However, quantitative analysis of pErk1/2 and pAkt in lysates of cells exposed to c29 together with 10 ng/ml NGF revealed a 2-fold increase in phosphorylated proteins, relative to cells treated with 10 ng/ml NGF and scrambled peptide. Indeed, the relative protein-band densities of pErk1/2 and pAtk in the former case were similar to those of 100 ng/ml NGF-treated controls (FIG. 6A, B). These results are consistent with the biological effects observed in the neurite outgrowth assays.

Taken together the above findings demonstrate that c29 has no obvious innate trophic activity but rather increases the sensitivity of PC12 cells to NGF via a Trk-specific mechanism. This in turn enhances NGF-activated differentiation and survival signalling pathways.

### Example 14

### c29 Peptide Acts Synergistically with TrkB

Given that the c29 peptide could pull down TrkB as well as TrkA (FIG. 4D), and that a recent study reported that p75^{ICD} could activate ERK1/2 signalling in TrkB- as well as TrkA-expressing cells (Ceni et al., 2010; Kommaddi et al., 2011b), we next investigated the actions of c29 in primary motor neuron cultures which do not express TrkA, but depend on TrkB activation via BDNF for survival (Koliatsos et al., 1993).

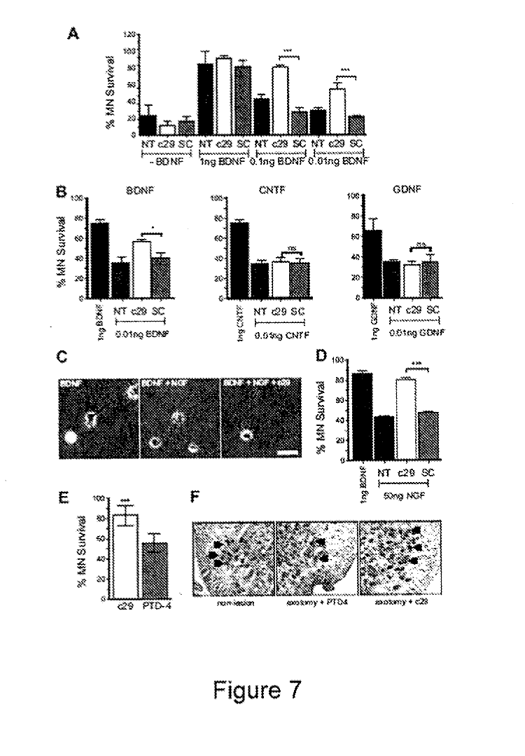

To determine whether c29 could potentiate BDNF survival signalling in a fashion similar to NGF- and TrkA-mediated mechanisms in PC12 cells, motor neuron cultures were grown in BDNF concentrations ranging from 0.01 to 10 ng/ml, and added c29 or scrambled peptide to the cells with fresh medium exchange on days 2 and 4. Analysis of motor neuron survival rates after 5 days revealed that, in comparison to control cultures, motor neurons grown in the presence of BDNF combined with c29 showed increased survival at the lower doses of BDNF (0.01-1.0 ng/ml), with maximal survival at 1 ng/ml (FIG. 7A). This result suggested that c29 was able to potentiate TrkB-dependent signalling via BDNF in motor neurons, similar to NGF-dependent and TrkA-mediated survival in PC12 cells.

To establish whether the effect of c29 was mediated specifically through ligand activated TrkB receptors, we analysed survival rates after withdrawal of growth factors that do not act on TrkB, in this case ciliary neurotrophic factor (CNTF) or glial-derived neurotrophic factor (GDNF). 24 hours after plating motor neurons in BDNF-, CNTF- or GDNF-containing medium, the cultures were transferred into medium containing a 10-fold lower concentration of the appropriate growth factor and then treated with c29 or scrambled peptide. In motor neuron cultures undergoing BDNF withdrawal, the presence of c29 resulted in significantly increased neuronal survival when compared to cultures treated with scrambled peptide (FIG. 7B). However c29 had no effect on the survival of motor neurons following CNTF or GDNF withdrawal (FIG. 7B), suggesting that coincident TrkB activation was required for c29 to be effective.

Motor neuron death can also be induced through the application of NGF (Wiese et al., 1999). It has been proposed that in this paradigm a higher concentration of NGF (50 ng/ml) compared to survival-promoting BDNF (1 ng/ml) can monopolise the activation of p75^{NTR} and thereby reduce its ability to form high-affinity receptors together with the TrkB receptor. As a result, the neurons undergo reduced trophic signalling that either leads to a situation of growth factor withdrawal and/or fails to override p75^{NTR}-mediated cell death signals (Bilderback et al., 2001; Sendtner et al., 1992; Wiese et al., 1999; Yoon et al., 1998). Using this paradigm, motor neurons grown in 1 ng/ml BDNF were significantly protected from NGF-induced cell death when treated with c29, whereas the scrambled peptide had no effect (FIG. 7C, D).

We next investigated whether c29 could prevent motor neuron death following growth factor withdrawal in vivo using a neonatal rat axotomy model. Following unilateral median and ulnar nerve transections of postnatal day 4 (P4) rat pups, c29 or the vehicle protein transduction domain (PTD4) was applied to the proximal site of transaction in a small cube of gel foam. After a 5-day recovery, the number of surviving C7-8 spinal cord motor neurons relative to the contralateral side was determined by histological counts. Such an axotomy causes 50% of the ipsilateral motor neurons to die (Cheema et al., 1996), with control peptide treatment having no effect on this degree of cell death (FIG. 7E, F). However, on average only 12% of the axotomised neurons treated with c29 died (FIG. 7E, F).

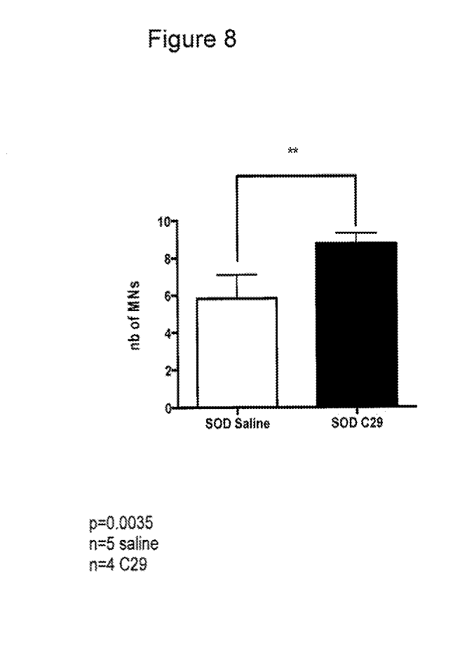

To determine whether c29 could reduce neuron death in a motor neuron mouse model, 60 day postnatal SOD1G93A mutant mice were injected subcutaneously by osmotic pump with 5 mg/kg of TAT-c29, scrambled peptide, or the peptide vehicle. After 115 days postnatal, neuron survival in the dorsal horn was determined. The results are shown in FIG. 8. As can be seen from FIG. 8, treatment of mice with TAT-c29 results in a 30% significant enhancement of motor neuron survival to equivalent of wild-type motor neuron number (10 motor neurons per section per dorsal horn) by the time of clinical disease onset.

Taken together these results indicate that c29 can potentiate neuronal survival by facilitating Trk function both in vitro and in vivo.

### Example 15

### The Juxtamembrane Region of p75^{NTR} Alters the Association of NGF with TrkA

Thus far we had observed that p75^{ICD} overexpression or generation of the endogenous p75^{ICD} resulted in enhanced neurite outgrowth and Erk1/2 activation. A smaller peptide fragment, c29, could similarly potentiate neurite outgrowth and Trk-mediated trophic signalling, as well as rescue neurons from growth-factor withdrawal in vitro and in vivo. In addition, this juxtamembrane region of p75^{NTR} was found to be both necessary and sufficient to mediate p75^{NTR}-Trk interactions. Based on the report that the juxtamembrane domain of p75^{NTR} is required for the generation of high-affinity NGF receptors (Esposito et al., 2001), we tested whether c29 was acting by increasing the binding rate of NGF to TrkA. In order to investigate the binding of NGF to cells expressing various combinations of neurotrophin receptors we used fluorescently labelled NGF (NGF-FITC), and flow cytometry analysis of ligand binding. The advantage of using flow cytometry, rather than the more traditional method based on radiolabelled NGF, is its ability to assess ligand binding in real time, on a per cell as well as total population basis (Bednar et al., 1997; Fay et al., 1991).

To test the extent of NGF binding we first incubated HEK293 cells transiently transfected with TrkAK538A, p75^{FL} or p75^{Δ-JUX} with excess NGF-FITC (26 nM) for 60 minutes (to reach receptor saturation). Although experiments were conducted on ice (to prevent endocytosis), we also utilised the TrkAK538A construct to ensure any effects on binding were not the result of activation of TrkA, e.g. promotion of p75^{NTR} cleavage. We found that in each condition, 80% of cells (the approximate transfection rate) bound sufficient NGF-FITC to have a fluorescence intensity above background (non-transfected cells incubated with NGF-FITC) with no statistical differences observed between the different expression constructs (Table 1). Furthermore, there was no significant difference in the mean, median or maximal fluorescence per cell in these populations (Table 1).

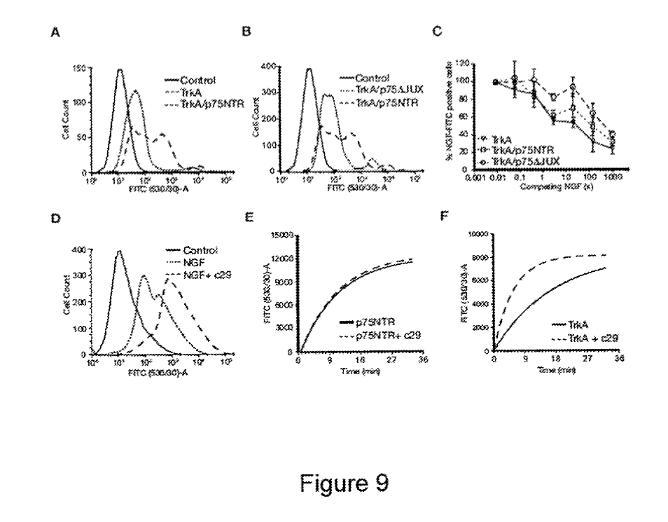

To confirm that the juxtamembrane domain was required to mediate the increased binding of NGF to TrkA-expressing cells (Esposito et al., 2001), p75^{FL} or p75^{Δ-JUX} was cotransfected with TrkAK538A, and the cells were incubated with NGF-FITC as above. The population of cells expressing both TrkAK538A and p75FL contained 12% more cells with a fluorescence intensity above background compared to the population expressing TrkAK538A alone (p=0.005; FIG. 9A; Table 1). The mean (p=0.009) and maximal (p=0.002) fluorescence of the population was also significantly increased (FIG. 9A; Table 1). This indicated that coexpression of p75^{FL} can increase the affinity of TrkA-expressing cells. The results demonstrate that TrkA does not need to be activated or endocytosed in order for p75^{NTR} to modulate NGF binding.

Interestingly, the population of cells expressing both p75^{FL} and TrkK538A had a significant increase in the median (p=0.019), mean (p=0.0004) and maximum (p=0.005) fluorescence compared to the cell population co-expressing p75^{Δ-JUX} with TrkK538A (Table 1). This was partially due to the surprising observation that the fluorescence parameters of cells expressing both p75^{Δ-JUX} and TrkAK538A were significantly lower than those of cells expressing both p75^{FL} and TrkAK538A or TrkK538A alone (63%±9.4; p=0.0013; FIG. 9A; Table 1). However, p75^{Δ-JUX} only-expressing cells had equivalent NGF-FITC binding to that of cells transfected with either p75^{FL} or TrkAK538A alone (p=0.38; Table 1), and there was no significant change in the number of cells in the population expressing both p75^{Δ-JUX} and TrkAK538A that bound significant NGF-FITC compared to the population of cells expressing both p75^{FL} and TrkAK538A.

TABLE 1

| | | | | | Mean | | | |
|---|---|---|---|---|---|---|---|---|
| | % of cell | | | | fluorescence | | Maximal | |
| | population with | | Median | | per | | fluorescence | |
| | FITC above | | population | | FITC-positive | | per cell of the | |
| Treatment | background | Statistics | fluorescence | Statistics | cell | Statistics | population | Statistics |
| p75^{FL} | 67.0 ± 1.2% | P = 0.20 | 4821 ± 2121 | P = 0.38 | 7088 ± 2172 | P = 0.48 | 5214 ± 1493 | P = 0.99 |
| n = 6 | | c.f. p75 [symbol] | | c.f. p75 [symbol] | | c.f. p75 [symbol] | | c.f. p75 [symbol] |
| p75 [symbol] | 79.2 ± 5.6% | P = 0.11 | 5210 ± 1680 | P = 0.32 | 8029 ± 2026 | P = 0.43 | 5210 ± 1051 | P = 0.27 |
| n = 3 | | c.f. TrkA [symbol] | | c.f. TrkA [symbol] | | c.f. TrkA [symbol] | | c.f. TrkA [symbol] |
| TrkA [symbol] | 73.0 ± 5.2% | p = 0.31 | 5027 ± 2047 | P = 0.27 | 7861 ± 3104 | P = 0.45 | 8482 ± 4612 | P = 0.12 |
| n = 9 | | c.f. p75^{FL} | | c.f. p75^{FL} | | c.f. p75^{FL} | | c.f. p75^{FL} |
| TrkA [symbol] + p75^{FL} | 112 ± 4.8% n = 3 | P = 0.005 | 119 ± 22.0% | P = 0.101 | 111.7 ± 5.2% | P = 0.009 | 134.3 ± 9.6% | P = 0.002 |
| c.f. TrkA [symbol] | | | | | | | | |
| n = 3 | | | | | | | | |
| TrkA [symbol] + p75 [symbol] | 103 ± 10% n = 3 | P = 0.29 | 63.8 ± 9.4% | P = 0.0013 | 92.8 ± 4.7% | P = 0.027 | 91.9 ± 1.7% | P = 0.0006 |
| c.f. TrkA [symbol] | | | | | | | | |
| n = 3 | | | | | | | | |
| TrkA [symbol] + p75^{FL} | 109.4 ± 13.3% | P = 0.15 | 191.4 ± 52.1% | P = 0.019 | 120.5 ± 3.8% | P = 0.0004 | 123.4 ± 8.8% | P = 0.006 |
| c.f. TrkA [symbol] + p75 [symbol] | | | | | | | | |
| n = 3 | | | | | | | | |
| p75^{FL} ± n29 c.f. | 100.5 ± 14.2% | P = 0.096 | 103.8 ± 11.2% | P = 0.29 | 105.2 ± 3.6% | P = 0.071 | 98.7 ± 4.3% | P = 0.32 |
| p75^{FL} | | | | | | | | |
| n = 3 | | | | | | | | |
| TrkA [symbol] + c29 c.f. | 112.2 ± 5.3% | P = 0.008 | 140.5 ± 21.3% | P = 0.015 | 125.0 ± 12.1% | P = 0.012 | 128.0 ± 11.7% | P = 0.007 |
| TrkA [symbol] | | | | | | | | |
| n = 3 | | | | | | | | |
| PC12 | 67.3 ± 12.0% n = 3 | | 6313 ± 1383 | | 7712 ± 1148 | | 17308 ± 5401 | |
| n = 3 | | | | | | | | |
| PC12 + SC | 100.3 ± 5.1% | P = 0.20 | 97.0 ± 3.8% | P = 0.12 | 95.3 ± 7.4% | P = 0.22 | 100.4 ± 9.6% | P = 0.47 |
| c.f. PC12 | | | | | | | | |
| n = 3 | | | | | | | | |
| PC12 + c29 | 115.3 ± 13.4% | P = 0.08 | 122.5 ± 8.4% | P = 0.004 | 121.4 ± 5.5% | P = 0.001 | 123.1 ± 5.2% | P = 0.0008 |
| c.f. PC12 + SC | | | | | | | | |
| n = 3 | | | | | | | | |
| PC12 + c29 | 115.7 ± 19.7% | P = 0.12 | 127.4 ± 8.1% | P = 0.002 | 126.3 ± 4.8% | P = 0.0003 | 123.6 ± 17.3% | P = 0.038 |
| c.f. PC12 | | | | | | | | |
| n = 3 | | | | | | | | |

[symbol]

indicates data missing or illegible when filed

These results indicate that the p75^{Δ-JUX} protein is significantly impairing NGF binding to cells co-expressing TrkA, an observation that paralleled our functional results in which both the p75^{Δ-JUX} and p75^{NGLY} proteins inhibited PC12 cell neurite growth (FIG. 3A, B) and Erk1/2 activation (FIG. 3C) of PC12 cells. This also suggests that, in cells expressing TrkAK538A and p75^{FL}, the observed increase in population fluorescence levels was not merely due to an increase in the number of Individual neurotrophin receptors being expressed on the plasma membrane of individual cells.

We next performed ligand-binding competition experiments to measure the dissociation of NGF-FITC from TrkAK538A expressing cells. Cells were incubated with 26 nM NGF-FITC for 60 minutes, before addition of unlabelled NGF at concentrations ranging from 100 pM to 10 μM, as a 1000 fold excess NGF can compete all NGF from TrkA expressing cells (Hempstead et al., 1991) for a further 30 minutes, after which the amount of NGF-FITC bound to the cells was analysed by flow cytometry.

The amount of bound NGF-FITC (median fluorescence of the positive population) was calculated as a percentage of the control condition in which cells were incubated with NGF-FITC for 60 minutes. Analysis of cells expressing p75^{Δ-JUX}, p75^{FL}, or TrkAK538A revealed a significant decrease in NGF-FITC binding to the cell populations as the concentration of unlabelled NGF increased. The concentration of unlabelled NGF required for NGF-FITC binding to fall to background fluorescence levels was 1000 fold excess for cell populations expressing any combination of receptors, with no significant difference in the competition curves for cells expressing TrkAK538A alone, p75^{NTR} and TrkAK538A or p75^{Δ-JUX} and TrkAK538A (FIG. 9C). These data indicate that the dissociation rate of NGF for TrkAK538A is not significantly altered by co-expression of p75^{NTR}, in line with previous reports (Mahadeo et al., 1994). Off rates were therefore not considered a significant factor in the observed increase in population fluorescence due to p75^{FL} co-expression.

Next, to test if the juxtamembrane domain of p75^{NTR} alone could alter NGF binding, the effect of the cell-permeable c29 peptide on NGF binding was determined. Nontransfected cells incubated with c29 (FIG. 9D) or scrambled peptide and NGF-FITC had only background levels of fluorescence (Table 1), indicating that the peptides did not affect the treated cells innate ability to interact with NGF. Furthermore, the fluorescence profile of the population of HEK293 cells transfected with p75^{NTR} and treated with NGF-FITC did not change if cells were pre-treated with the c29 peptide (Table 1). By contrast, the total number of fluorescent cells (p=0.008) as well as the median (p=0.015), mean (p=0.012) and maximal fluorescence (p=0.007) of the population of the c29-treated TrkAK538A-transfected population were significantly increased compared to those of control TrkAK538A-transfected cells (FIG. 9D; Table 1). Similarly, when the experiment was performed using PC12 cells, all measured fluorescence parameters of the FITC-positive population of c29-treated TrkAK538A-transfected cells were increased compared to those of control TrkAK538A-transfected cells or scrambled peptide-treated TrkAK538A-transfected cells (p<0.001 for all parameters; Table 1).

These results indicate that c29 is sufficient to increase the ability of TrkA-expressing cells to bind NGF.

To determine whether the increase in the amount of NGF-FITC bound per cell was due to an increased association rate, we also measured the binding of NGF-FITC to populations of TrkAK538A- and p75^{NTR}-expressing HEK293 cells over time. As reported previously (Hempstead et al., 1991; Hempstead et al., 1990), we found that binding of NGF-FITC to p75^{FL}-expressing HEK293 cells (half time of 7.2 minutes, average of 3 experiments) occurred at a faster rate than for TrkA-expressing cells (half time of 11 minutes, average of 3 experiments). For p75^{NTR}-expressing cells, the time it took for the mean fluorescence per cell of the population to plateau following NGF-FITC treatment was not discernably different between untreated cells and those treated with c29 (half time of 7.1 minutes; FIG. 9E).

By contrast, our experiments revealed that the rate of uptake of NGF-FITC was considerably faster in the population of TrkAK538A-expressing cells pre-treated with c29 (half time of 3.6 minutes, average of 3 experiments) than the rate observed for untreated TrkAK538A-expressing cells (FIG. 9F). Comparison of the binding curves derived from these data (based on the best fit with a Hill-Slope association curve for single receptor ligand binding) revealed that c29-treated TrkAK538A-expressing cells not only had a ˜3-fold faster association rate than untreated TrkAK538A-expressing cells, but the mean fluorescence of the population following receptor saturation (˜20 minutes after NGF addition) was at least 20% higher when TrkAK538A-expressing cells were treated with c29 (p<0.01 ANOVA), correlating with binding at the 1 hour time point, (FIG. 9F; see also Table 1). These data suggest that the number of receptor sites capable of binding NGF, as well as the association rate, is increased when cells contain c29.

### Example 16

### c29 Acts by an Allosteric Mechanism

The significantly higher mean fluorescence following receptor saturation of TrkA expressing cells in the presence of c29 or p75^{NT} was indicative of the cells containing more receptor binding sites. Therefore, we next tested whether the mechanism by which c29 was affecting NGF binding kinetics and function was by altering the number of the TrkA (or p75^{NTR}) receptors at the plasma membrane.

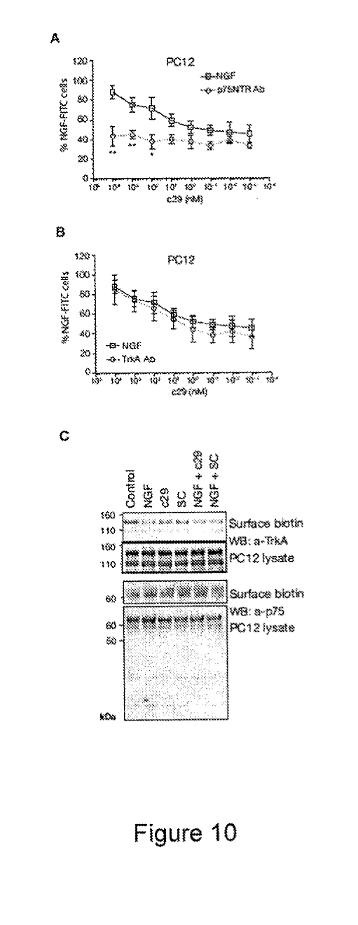

First, to determine the concentration of c29 required to modulate NGF binding, c29 levels were titrated with a fixed concentration of NGF-FITC. An increase in the median level of fluorescence in the population c29-treated population compared to the control population was observed at 1 pM c29, with significant effects measured at c29 concentrations between 100 pM and 1 nM (FIG. 10A).

Next, the level of surface receptor expression was analysed using extracellular antibodies specific to the NGF binding domains of either p75^{NTR} or TrkA in the presence of increasing concentrations of c29. In p75^{NTR}-expressing cells, c29 had no effect on the extent of antibody binding (FIG. 10B). However, increasing the concentration of c29 applied to cells prior to TrkA antibody incubation resulted in a significant increase in antibody binding (with the effect beginning between 100 pM and 1 nM c29), a change that mirrored the binding of NGF to the cells (FIG. 10B).

However, analysis using surface biotinylation revealed no significant difference in either surface or total receptor levels of either TrkA and p75^{NTR} in PC12 cells, regardless of whether the cells were treated individually with NGF, 1 μM c29 or scrambled peptide or if they were treated with both NGF and c29 or scrambled peptide (FIG. 10C). Similarly the rate of production of endogenous p75^{CTF} or p75^{ICD} fragments in p75^{FL}-transfected HEK293 cells did not differ significantly between c29- and scrambled peptide-treated cells or untreated cells (data not shown). Together these data argue that there is a significant increase in the number of receptor sites capable of binding NGF when cells contain c29, even though the number of receptors per se on the plasma membrane of individual cells was not significantly affected by c29.

### Example 17

### c29 Peptide Enhances Memory Acquisition and Extinction

To determine the effect of c29 on memory acquisition and extinction, mice were treated with a single infusion of the prefrontal cortex with 1 μM biotin-TAT-c20 peptide, scampled peptide, or the peptide vehicle, and subjected to fear conditioning. Fear conditioning was induced by standard methods, as previously described: Activation of BDNF signaling prevents the return of fear in female mice. Baker-Andresen et al. Learn Mem. 2013 Apr. 15; 20(5):237-40. Briefly, in fear conditioning, mice were given a foot shock at the same time as hearing a tone if they enter into a specific part of a grid (a conditioning stimulus). One day later, when the animals hear the tone but are not given the shock, they demonstrate a fear response. That is, they freeze and do not move. The duration of this response was timed and % of freeze response calculated. Enhanced freezing is a measurement of enhanced learning or memory acquisition or retrieval.

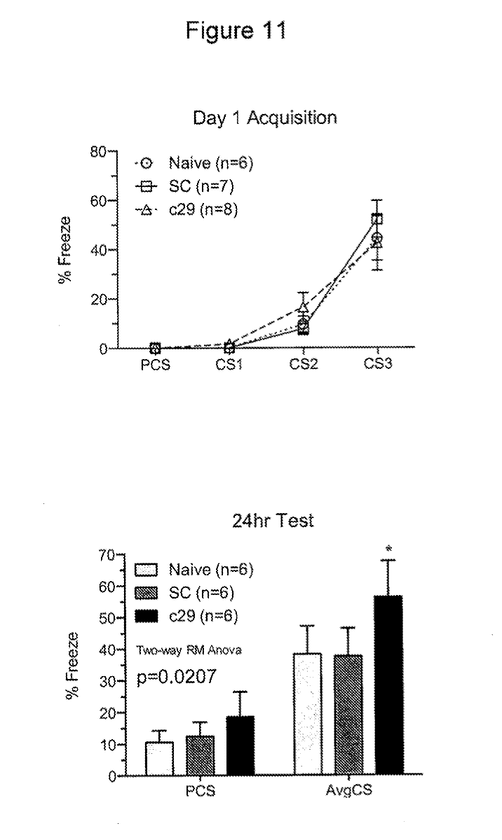

A conditioning stimulus was applied on day 1, 2 and 3, and the % freeze response for the mice was determined. The results are shown in FIG. 11A. As can be seen from FIG. 11A, administration of biotin-TAT-c29 showed no obvious effect on the animals ability to learn (memory acquisition).

To determine the effect of c29 on memory, mice were subjected to fear conditioning as above, and after 24 hours, their ability to remember was tested. The results are shown in FIG. 11B. The results in FIG. 11B show that a single infusion of the prefrontal cortex of mice with biotin-TAT-c29 peptide at a dose of 1 μM enhances memory acquisition, specifically, assisting the retention of a conditioned fear memory, a process known to be mediated by brain-derived neurotrophic factor (BDNF)-TrkB signaling. Thus, by administering biotin-TAT-c29 peptide, the animals have enhanced memory acquisition.

### Example 18

### c29 Peptide Enhances Memory Extinction

In vivo studies (n=4) have shown that a single infusion of the prefrontal cortex of mice with biotin-TAT-c29 peptide at a dose of 1 μM enhances memory extinction, specifically, assisting the removal of a conditioned fear memory, a process known to be mediated by brain-derived neurotrophic factor (BDNF)-TrkB signaling.

Following fear conditioning (Example 17), exposure to the tone without the shock is termed extinction (removal of the memory). By administering the biotin-TAT-c29 peptide, the researchers have found that the animals have enhanced extinction.

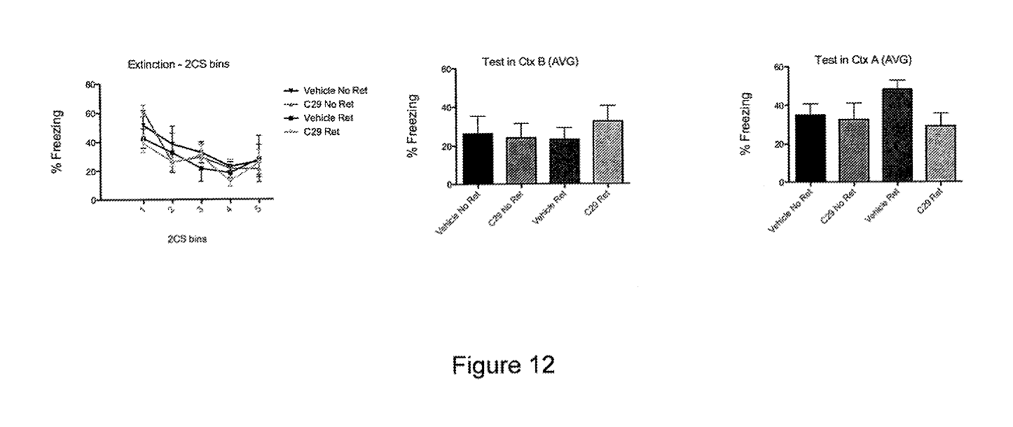

As shown in FIG. 12, there is a significant difference between VEH RET and C29 In fear conditioning, animals are given a foot shock at the same time as hearing a tone if they enter into a specific part of a grid. One day later, when these animals hear the tone but are not given the shock, they demonstrate a fear response. That is, they freeze and do not move. Researchers can time the duration of this response. Repeated exposure to the tone without the shock is termed extinction (removal of the memory). By administering the biotin-TAT-c29 peptide, the researchers have found that the animals have enhanced extinction.

As shown in FIG. 10, there is a significant difference between VEH RET and C29 RET. Effectively, c29, when infused into the pre-frontal cortex, prevents the return of fear normally observed in female mice when presented with a retrieval cue prior to extinction training.

The fear response induced through fear conditioning is very similar to anxiety in humans. Accordingly, fear conditioning is a widely used experimental model for a range of disorders including post-traumatic stress disorder, panic attacks. Further, fMRI imaging studies have confirmed that these disorders share the same pattern of brain activity as seen during fear conditioning. Therefore, the results shown here strongly indicate that c29 could be used to treat anxiety and disorders such as post-traumatic stress disorder and panic attacks in humans.

Together, these results suggest that c29 can act to enhance learning, either a novel task (fear learning) or as memory updating (fear extinction). This is also known as plasticity.

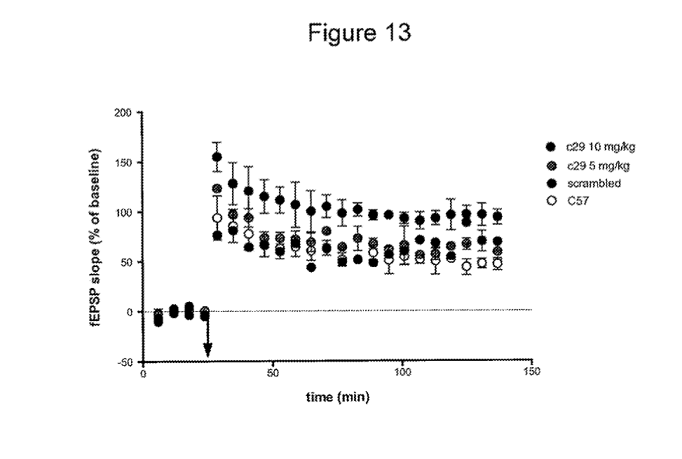

In further support of this, LTP was induced in mice by standard methods, as previously described: Kameda et al. Transl Psychiatry. 2012 Jan. 31; 2:e72. Activation of latent precursors in the hippocampus is dependent on long-term potentiation. The results are shown in FIG. 13. In this regard, FIG. 13 shows that a single infusion of TAT-c29 by i.p injection results in enhanced long term potentiation (LTP), and physiological change thought to be the best representation of a physical embodiment of a new memory. BDNF acting via TrkB is well established to enhance this type of synaptic activity, including specifically hippocampal late-phase LTP at CA1-CA3 synapses (as measured here), and mice with reduced BDNF secretion show deficits in this LTP (e.g. Y. Lu et al., 2008; An et al., 2008; Ninan et al., 2010). As shown in FIG. 13, Mice infused with 10 mg/kg TAT-c29 have enhanced LTP (p<0.001) in the hippocampus in vivo in response to a high frequency stimulus compared to LTP induced in scrambled peptide- or vehicle-treated animals (C57). Also, mice infused with 5 mg/kg TAT-c29 I.P also have a transient enhancement of LTP lasting only the first 6 minutes (p<0.05) after the high frequency train. In FIG. 13, c29 10 mg/kg: n=3 animals; c29 5 mg/kg: n=2 animals; scrambled 10 mg/kg: n=3 animals, c57: n=2 animals.

### Example 19

### Functional Fragments of c29

The c29 peptide was labelled with biotin and attached to a sepharose bead column. Lysates from cells was passed over the column followed by several wash steps. The proteins that bound to the c29 peptide (proteins that are pulled down) were eluted from the column with SDS buffer and separated in size by SDS-PAGE electrophoresis. The gel was Western blotted and the membrane probed with antibodies to proteins of interest.

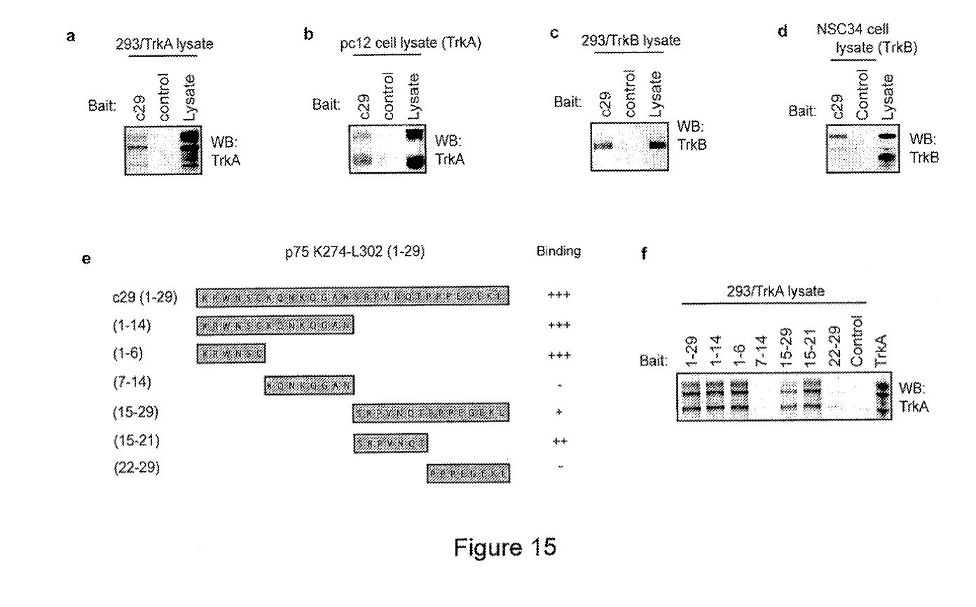

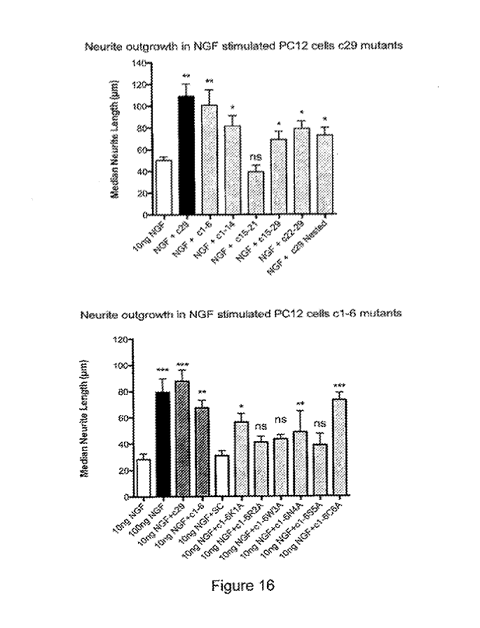

As shown in FIG. 15F, fragments (1-14), (1-6), (15-29), and (22-29) clearly bind TrkA. Fragments were incubated with PC12 cells and NGF to test for the ability to promote neurite growth. As shown in FIG. 16A, c29, c1-6, c1-14, c15-29 and c22-29 stimulated neurite growth. Mutants of c1-6 were tested for the ability to stimulate neurite growth. As shown in FIG. 16B, mutants c1-6K1A, c1-6N4A and c1-6C6A (ARWNSC, KRWASC, and KRWASA, respectively) stimulated neurite growth.

### REFERENCES

An J J, Gharami K, Liao G Y, Woo N H, Lau A G, Vanevski F, Torre E R, Jones K R, Feng Y, Lu B, Xu B (2008) Cell 134:175-187.
Baker-Andresen D, Flavell C R, Li X, Bredy T W (2013) Learn Mem 20:237-240.
Barrett G L, Georgiou A (1996) J Neurosci Res 45:117-128.
Bednar B, Cunningham M E, McQueney P A, Egbertson M S, Askew B C, Bednar R A, Hartman G D, Gould R J (1997) Cytometry 28:58-65.
Bibel M, Hoppe E, Barde Y A (1999) EMBO J 18:616-622.
Bilderback T R, Gazula V R, Dobrowsky R T (2001) J Neurochem 76:1540-1551.
Ceni C, Kommaddi R P, Thomas R, Vereker E, Liu X, McPherson P S, Ritter B, Barker P A (2010) J Cell Sci 123:2299-2307.
Chao M V, Hempstead B L (1995) Trends Neurosci 18:321-326.
Cheema S S, Barrett G L, Bartlett P F (1996) J Neurosci Res 46:239-245.
Cheema S S, Richards L, Murphy M, Bartlett P F (1994) J Neurosci Res 37:213-218.
Coulson E J, Reid K, Barrett G L, Bartlett P F (1999) J Biol Chem 274:16387-16391.
Coulson E J, Reid K, Baca M, Shipham K A, Hulett S M, Kilpatrick T J, Bartlett P F (2000) J Biol Chem 275:30537-30545.
Esposito D, Patel P, Stephens R M, Perez P, Chao M V, Kaplan D R, Hempstead B L (2001) J Biol Chem 276:32687-32695.
Fay S P, Posner R G, Swann W N, Sklar L A (1991) Biochemistry (Mosc) 30:5066-5075.
Green S H, Rydel R E, Connolly J L, Greene L A (1986) J Cell Biol 102:830-843.
Greene L A (1978) J Cell Biol 78:747-755.
Greene L A, Tischler A S (1976) Proc Natl Acad Sci USA 73:2424-2428.
Greene L A, Shain W, Chalazonitis A, Breakfield X, Minna J, Coon H G, Nirenberg M (1975) Proc Natl Acad Sci USA 72:4923-4927.
Hartmann M, Brigadski T, Erdmann K S, Holtmann B, Sendtner M, Narz F, Lessmann V (2004) J Cell Sci 117:5803-5814.
Hempstead B L, Patil N, Thiel B, Chao M V (1990) J Biol Chem 265:9595-9598.
Hempstead B L, Martin-Zanca D, Kaplan D R, Parada L F, Chao M V (1991) Nature 350:678-683.
Ho A, Schwarze S R, Mermelstein S J, Waksman G, Dowdy S F (2001) Cancer Res 61:474-477.
Huber L J, Chao M V (1995) J Neurosci Res 40:557-563.
Inagaki N, Thoenen H, Lindholm D (1995) Eur J Neurosci 7:1125-1133.
Jing S, Tapley P, Barbacid M (1992) Neuron 9:1067-1079.
Kameda M, Taylor C J, Walker T L, Black D M, Abraham W C, Bartlett P F (2012) Transl Psychiatry 2:e72.
Kaplan D R, Stephens R M (1994) J Neurobiol 25:1404-1417.
Kommaddi R P, Dickson K M, Barker P A (2011a) J Neurochem 116:396-405.
Kommaddi R P, Thomas R, Ceni C, Daigneault K, Barker P A (2011 b) The FASEB 25:2061-2070.
Lu Y, Christian K, Lu B (2008) Neurobiol Learn Mem 89:312-323.
Mahadeo D, Kaplan L, Chao M V, Hempstead B L (1994) J Biol Chem 269:6884-6891.
Matusica D, Fenech M P, Rogers M L, Rush R A (2008) J Neurosci Res 86:553-565.
Ninan I, Bath K G, Dagar K, Perez-Castro R, Plummer M R, Lee F S, Chao M V (2010) J Neuroscience 30:8866-8870.
Qui M S, Green S H (1992), Neuron 9:705-717.
Sendtner M, Holtmann B, Kolbeck R, Thoenen H, Barde Y A (1992) Nature 360:757-759.
Shaw G, Morse S, Ararat M, Graham F L (2002) FASEB 16:869-871.
Traverse S, Gomez N, Paterson H, Marshall C, Cohen P (1992) Biochem J 288 (Pt 2):351-355.
Underwood C K, Reid K, May L M, Bartlett P F, Coulson E J (2008) Mol Cell Neurosci 37:346-358.
Vaudry D, Stork P J, Lazarovici P, Eiden L E (2002) Science 296:1648-1649.
Vesa J, Kruttgen A, Shooter E M (2000) J Biol Chem 275:24414-24420.
Wiese S, Metzger F, Holtmann B, Sendtner M (1999) Eur J Neurosci 11:1668-1676.
Wiese S, Herrmann T, Drepper C, Jablonka S, Funk N, Klausmeyer A, Rogers M L, Rush R, Sendtner M (2010) Nat Protoc 5:31-38.
Wolf D E, McKinnon C A, Daou M C, Stephens R M, Kaplan D R, Ross A H (1995) J Biol Chem 270:2133-2138.
Yang T T, Sinai P, Kain S R (1996) Anal Biochem 241:103-108.
Yano H, Lee F S, Kong H, Chuang J, Arevalo J, Perez P, Sung C, Chao M V (2001) J Neurosci 21:RC125.
Yoon S O, Casaccia-Bonnefil P, Carter B, Chao M V (1998) J Neurosci 18:3273-3281.

1-47. (canceled)

48. A method of regulating neurotrophin-tyrosine kinase receptor (neurotrophin-Trk) signaling in a cell comprising modulating the interaction between the juxtamembrane region of the intracellular domain of the neurotrophin receptor p75 (p75ICD juxtamembrane region) and Trk.

49. A method according to claim 48, wherein the neurotrophin is selected from nerve growth factor (NGF), brain derived neurotrophic factor (BDNF), neurotrophin-3 (NT-3) and neurotrophin-4 (NT-4).

50. A method according to claim 48, wherein the tyrosine receptor kinase is selected from TrkA, TrkB and TrkC.

51. A method according to claim 48, wherein the neurotrophin-Trk signaling is NGF-TrkA signaling.

52. A method according to claim 48, wherein the neurotrophin-Trk signaling is BDNF-TrkB signaling or NT4-TrkB signaling.

53. A method according to claim 48, wherein the neurotrophin-Trk signaling is NT3-TrkC signaling.

54. A method according to claim 48, wherein modulation of the interaction between p75ICD juxtamembrane region and Trk is direct.

55. A method according to claim 54, wherein modulation of the interaction between p75ICD juxtamembrane region and Trk up-regulates neurotrophin-Trk signaling.

56. A method according to claim 55, wherein neurotrophin-Trk signaling is up-regulated by contacting a cell with a peptide, polypeptide or protein that corresponds to all or part of the p75ICD juxtamembrane region [SEQ ID NO: 1], or a functional derivative or homologue thereof.

57. A method according to claim 55, wherein neurotrophin-Trk signaling is up-regulated by contacting a cell with a peptide comprising an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 and SEQ ID NO: 12.

58. A method according to claim 55, wherein neurotrophin-Trk signaling is up-regulated by contacting a cell with the peptide of SEQ ID NO: 2 or SEQ ID NO: 7.

59. A method according to claim 54, wherein modulation of the interaction between p75ICD juxtamembrane region and Trk down-regulates neurotrophin-Trk signaling.

60. A method according to claim 59, wherein neurotrophin-Trk signaling is down-regulated by contacting a cell with (i) an antibody that binds specifically to the p75ICD juxtamembrane region [SEQ ID NO: 1] or a functional derivative or homologue thereof; (ii) an antibody that binds specifically to the region of Trk that binds the p75ICD juxtamembrane region or a functional derivative or homologue thereof; or (iii) a combination thereof.

61. A method according to claim 48, wherein modulation of the interaction between p75ICD juxtamembrane region and Trk is indirect.

62. A method according to claim 61, wherein modulation of the interaction between p75ICD juxtamembrane region and Trk is by modulating the endogenous levels of p75ICD juxtamembrane region in a cell.

63. A composition for use in modulating neurotrophin-Trk signaling comprising a compound that modulates the interaction between p75ICD juxtamembrane region and Trk.

64. A composition according to claim 63, wherein the compound comprises:
a. a peptide, polypeptide or protein that corresponds to all or part of the p75ICD juxtamembrane region [SEQ ID NO: 1], or a functional derivative or homologue thereof;
b. an antibody that binds specifically to the p75ICD juxtamembrane region [SEQ ID NO: 1], or a functional derivative or homologue thereof;
c. an antibody that binds specifically to the region of TrkB that binds the p75ICD juxtamembrane region;
d. a p75NTR α-secretase and/or p75NTR γ-secretase activator, or
e. a p75NTR α-secretase and/or p75NTR γ-secretase inhibitor.

65. A composition according to claim 63, wherein the composition comprises a peptide comprising, consisting essentially of, or consisting of the amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO 11 and SEQ ID NO: 12.

66. A composition according to claim 64, wherein the composition comprises the peptide of SEQ ID NO: 2 or 7.

67. A method of identifying a compound that up-regulates neurotrophin-Trk signaling comprising:
(i) providing a neurotrophin-Trk signaling system including Trk and p75ICD juxtamembrane region, under conditions which promote neurotrophin-Trk signaling;
(ii) contacting the system with a compound;
(iii) measuring the level of neurotrophin-Trk signaling;
(iv) contacting the neurotrophin-Trk signaling system with a candidate compound;
(v) measuring the level of neurotrophin-Trk signaling in the presence of the candidate compound; and
(vi) comparing the measured level of neurotrophin-Trk signaling in the presence of the candidate agent with the level of neurotrophin-Trk signaling in the absence of the candidate compound, wherein a statistically significant increase in the level of neurotrophin-Trk signaling in the presence of the candidate agent is indicative of a compound that up-regulates neurotrophin-Trk signaling.

68. A method according to claim 67, wherein the neurotrophin-Trk signaling system comprises cells that endogenously express a Trk which is activated by a neurotrophin and treating said cells with a neurotrophin and c29.

69. A method according to claim 67, wherein the neurotrophin-Trk signaling system comprises cells that are transfected with a construct comprising a gene encoding a Trk which is activated by a neurotrophin and treating said cells with a neurotrophin and c29.

70. A method of identifying a compound that down-regulates neurotrophin-Trk signaling comprising:
(i) providing a neurotrophin-Trk signaling system including Trk and p75ICD juxtamembrane region, under conditions which promote neurotrophin-Trk signaling;
(ii) contacting the system with a compound;
(iii) measuring the level of neurotrophin-Trk signaling;
(iv) contacting the neurotrophin-Trk signaling system with a candidate compound;
(v) measuring the level of neurotrophin-Trk signaling in the presence of the candidate compound; and
(vi) comparing the measured level of neurotrophin-Trk signaling in the presence of the candidate agent with the level of neurotrophin-Trk signaling in the absence of the candidate compound, wherein a statistically significant decrease in the level of neurotrophin-Trk signaling in the presence of the candidate agent is indicative of a compound that down-regulates neurotrophin-Trk signaling.

71. A method according to claim 70, wherein the neurotrophin-Trk signaling system comprises cells that endogenously express a Trk which is activated by a neurotrophin and treating said cells with a neurotrophin and c29.

72. A method according to claim 70, wherein the neurotrophin-Trk signaling system comprises cells that are transfected with a construct comprising a gene encoding a Trk which is activated by a neurotrophin and treating said cells with a neurotrophin and c29.

73. An assay for identifying a compound that modulates neurotrophin-Trk signaling comprising, comprising:
(i) measuring the level of p75ICD juxtamembrane region in a cell;
(ii) contacting the cell with a candidate compound;
(iii) measuring the level of p75ICD juxtamembrane region in the contacted cell of (ii); and
(iv) comparing the measured level of p75ICD juxtamembrane region of (i) and (iii), wherein a statistically significant difference in the level is indicative of a compound that modulates neurotrophin-Trk signaling comprising.

74. A method of treating a neurotrophin-Trk signaling related disease or disorder or a fear related disorder in a subject in need thereof, said method comprising administering to said subject a therapeutically effective amount of a composition that comprises a compound that modulates the interaction between p75ICD juxtamembrane region and Trk.

75. A method according to claim 74, wherein the method comprises administering to said subject a therapeutically effective amount of a composition that comprises a compound that up-regulates the interaction between p75ICD juxtamembrane region and Trk compound.

76. A method according to claim 74, wherein the compound that up-regulates the interaction between p75ICD juxtamembrane region and Trk compound is a peptide, polypeptide or protein that corresponds to all or part of the p75ICD juxtamembrane region [SEQ ID NO: 1], or a functional derivative or homologue thereof.

77. A method according to claim 74, wherein the disease or disorder is selected from the group consisting of cerebral palsy, trauma induced paralysis, vascular ischaemia associated with stroke, neural tumours, motoneurone disease, Parkinson's disease, Huntington's disease, Alzheimer's disease, multiple sclerosis and peripheral neuropathies associated with diabetes, heavy metal or alcohol toxicity, renal failure and/or infectious diseases such as herpes, rubella, measles, chicken pox, HIV and/or HTLV-1.

78. A method of promoting neuritre outgrowth in a subject in need thereof, said method comprising administering to said subject a therapeutically effective amount of a composition capable of up-regulating NGF-TrkA signaling.

79. A method according to claim 78, wherein the neurotrophin-Trk related disease or disorder is a psychiatric disorder.

80. A method of promoting memory extinction in a subject in need thereof, said method comprising administering to said subject a therapeutically effective amount of a composition comprising a compound that up-regulates the interaction between p75ICD juxtamembrane region and Trk.

81. A method according to claim 74, wherein the composition comprises a peptide comprising, consisting essentially of, or consisting of the amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO 11 and SEQ ID NO: 12.

82. A cognitive enhancer comprising a peptide, polypeptide or protein that corresponds to all or part of the p75ICD juxtamembrane region SEQ ID NO: 1.

83. A cognitive enhancer according claim 82, wherein the cognitive enhancer is a peptide comprising, consisting essentially of, or consisting of the amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 and SEQ ID NO: 12.

84. A cognitive enhancer according claim 82, wherein the cognitive enhancer is the peptide of SEQ ID NO: 2 or 7.

85. A cognitive enhancer according to claim 82, wherein the cognitive enhancer up-regulates neurotrophin-Trk signaling.

86. A cognitive enhancer according to claim 82, wherein the cognitive enhancer is used for the treatment of a disorder selected from post-traumatic stress disorder, panic attacks, schizophrenia, depression, bipolar spectrum disorder, anxiety, drug addiction, obsessive-compulsive disorder and Autism spectral disorder.

87. A method of diagnosing a neurotrophin-Trk signaling related disease or disorder in a subject suspected of having a neurotrophin-Trk signaling related disease or disorder, said method comprising detecting the level of expression of a gene encoding c29 polypeptide (a) in a test sample of tissue cells obtained from a subject, and (b) in a control sample of known normal tissue cells of the same cell type, wherein a higher or lower level of expression of said gene in the test sample as compared to the control sample is indicative of the presence of neurotrophin-Trk signaling related disease or disorder in a subject from which the test tissue cells were obtained.

88. A peptide comprising, consisting essentially of, or consisting of, an amino acid sequence selected from the group consisting of SEQ ID NO: 7, SEQ ID NO: 8, SEQ ID NO: 9, SEQ ID NO: 10, SEQ ID NO: 11 and SEQ ID NO: 12.

89. A method according to claim 79, wherein the composition comprises a peptide comprising, consisting essentially of, or consisting of the amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO 11 and SEQ ID NO: 12.

90. A method according to claim 80, wherein the composition comprises a peptide comprising, consisting essentially of, or consisting of the amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO 11 and SEQ ID NO: 12.
